(19) United States
(12) Patent Application Publication
(10) Pub. No.: US 2018/0055410 A1
(43) Pub. Date: Mar. 1, 2018

(54) STIMULATION AND SENSING SYSTEM FOR MERIDIAN CONDITION EVALUATION

(71) Applicant: Acumedical, Inc., North Oaks, MN (US)

(72) Inventors: Jiang Ding, Shoreview, MN (US); Lei Chen, North Oaks, MN (US); Jie Xia, Shoreview, MN (US)

(21) Appl. No.: 15/790,519

(22) Filed: Oct. 23, 2017

Related U.S. Application Data
(63) Continuation-in-part of application No. 15/270,754, filed on Sep. 20, 2016.
(60) Provisional application No. 62/381,912, filed on Aug. 31, 2016.

(51) Int. Cl. A61B 5/053; A61B 5/00; A61H 39/00; A61H 39/08

(52) U.S. Cl. CPC A61B 5/0532; A61H 39/086; A61H 39/002; A61B 5/4854

(57) ABSTRACT

A stimulation and sensing system may be configured to deliver an electrical signal to a patient and sense a response from one or more acupuncture points of the patient. The sensed response may be analyzed to detect a health state of the patient by evaluating a condition or property of the patient's meridian system. The electrodes may include noninvasive electrodes and/or percutaneous electrodes such as non-insulated and/or partially insulated acupuncture needles.

### CROSS REFERENCE TO RELATED APPLICATION

This application is a continuation-in-part of, and claims benefits to, U.S. patent application Ser. No. 15/270,754, filed on Sep. 20, 2016 and claiming the benefit of priority under 35 U.S.C. §119(e) of U.S. Provisional Patent Application Ser. No. 62/381,912, filed on Aug. 31, 2016, each of which is incorporated herein by reference in its entirety.

This application is related to U.S. patent application Ser. No. 15/270,779, filed on Sep. 20, 2016 and claiming the benefit of priority under 35 U.S.C. §119(e) of U.S. Provisional Patent Application Ser. No. 62/381,912, filed on Aug. 31, 2016, each of which is incorporated herein by reference in its entirety.

### TECHNICAL FIELD

This document relates generally to medical devices and more particularly to a stimulation and sensing system allowing from detecting health state of a person by evaluating conditions of the patient's meridian system.

### BACKGROUND

Acupuncture has been an important component of traditional Chinese medicine and increasingly accepted in Western countries as a form of alternative medicine. This growing acceptance of acupuncture is largely attributed to its therapeutic effects (e.g., effectiveness in pain relief) that can be achieved without substantial discomfort for the patient and difficulty for the practitioner. However, the fundamental theory of acupuncture remains mysterious because it has yet been proven by modern science. The lack of a scientific approach to the practice of acupuncture has prevented common application of acupuncture in many potentially effective areas beyond a few applications such as pain control, and has also prevented evaluation of effectiveness of an acupuncture therapy based on changes of tissue property at acupuncture points.

### SUMMARY

A stimulation and sensing system may be configured to deliver an electrical signal to a patient and sense a response from one or more acupuncture points of the patient. The sensed response may be analyzed to detect a health state of the patient by evaluating a condition or property of the patient's meridian system. The electrodes may include noninvasive electrodes and/or percutaneous electrodes such as non-insulated and/or partially insulated acupuncture needles.

In one embodiment, a system for detecting a health state in a patient may include a test signal generation circuit, a sensing circuit, and a control circuit. The test signal generation circuit may be configured to generate a test signal having a test signal amplitude (V_{S}) to be delivered to the patient. The sensing circuit may be configured to sense one or more response signals each representative of propagation of the test signal to one of one or more sensing points selected from acupuncture points of the patient. The control circuit may be coupled to the test signal generation circuit and the sensing circuit. The control circuit may include measurement circuitry and data processing circuitry. The measurement circuitry may be configured to measure a response to the delivery of the test signal. The response may include a voltage amplitude (V_{A}) of each sensed response signal of the one or more sensed response signals. The data processing circuitry may be configured to produce a meridian condition indicator using the V_{S} and the V_{A}.

In one embodiment, a method for detecting a health state in a patient is provided. The method can include delivering a test signal having a test signal amplitude (V_{S}) to the patient, sensing one or more response signals each representative of propagation of the test signal to one of one or more sensing points selected from acupuncture points of the patient, measuring a response to the delivery of the test signal, the response including a voltage amplitude (V_{A}) of each sensed response signal of the one or more sensed response signals, and producing a meridian condition indicator for the each sensing point based on the V_{S} and the V_{A}.

This summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

FIG. 1 is a block diagram illustrating an embodiment of a sensing and stimulation system.

FIG. 2 is a block diagram illustrating an embodiment of a central station of the monitoring and stimulation system.

FIG. 3 is a block diagram illustrating an embodiment of a substation of the monitoring and stimulation system.

FIG. 4 is a block diagram illustrating an embodiment of an input/output device (IOD) of the monitoring and stimulation system.

FIG. 5 is a block diagram illustrating an embodiment of a docking station of the monitoring and stimulation system.

FIG. 6 is an illustration of an embodiment of an acupuncture needle.

FIG. 7 is an illustration of an embodiment of a partially insulated acupuncture needle.

FIG. 8 is an illustration of another embodiment of a partially insulated acupuncture needle.

FIG. 9 is an illustration of an embodiment of an acupuncture needle including an electrical connector.

FIG. 10 is an illustration of an embodiment of acupuncture needles connected to a medical device, such as the IOD of FIG. 1 or 4, and inserted in tissue.

FIG. 11 is an illustration of an embodiment of an acupuncture needle and a guiding tube with an electrode.

FIG. 12 is an illustration of an embodiment of the acupuncture needle and the guiding tube of FIG. 11 connected to a medical device, such as the IOD of FIG. 1 or 4, and inserted into tissue.

FIG. 13 is an illustration of an embodiment of an acupuncture needle including a needle stopper with an electrode.

FIG. 14 is an illustration of an embodiment of the acupuncture needle of FIG. 13 connected to a medical device, such as the IOD of FIG. 1 or 4, and inserted into tissue.

FIG. 15 is an illustration of an example of distribution of an electric field produced by a dipole in a uniform dielectric medium.

FIG. 16 is an illustration of an example of electric field of a dipole in a healthy human body.

FIG. 17 is an illustration of an example of electric field of a dipole in a diseased human body.

FIG. 18 is an illustration of an example of deviation of electric potentials measured from acupuncture points distant from a dipole in a patient.

FIG. 19 is a block diagram illustrating an embodiment of a system including a meridian condition tester and electrodes for detecting a health state of a patient.

FIG. 20 is a block diagram illustrating an embodiment of electrodes with leads for detecting a health state of a patient, such as for use in the system of FIG. 19.

FIG. 21 is an illustration of an embodiment of electrodes for applying a test signal and measuring amplitude of a source voltage, such as for use in the system of FIG. 19.

FIG. 22 is a block diagram illustrating an embodiment of a meridian condition tester, such as the meridian condition tester of FIG. 19.

FIG. 23 is a flow chart illustrating an embodiment of a method for detecting a health state of a patient.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

This document discusses, among other things, methods and apparatuses for performing various medical diagnostic and/or therapeutic functions by incorporating modern technologies in acupuncture and/or applying various aspects of acupuncture in medical electronic devices.

The original acupuncture concept was based on the view of traditional Chinese medicine and philosophy that forces of “yin” and “yang” exist everywhere in the nature, where every natural phenomenon, including biological phenomenon, results from their constant interactions. Yin (dark) represents feminine, passive, or accepting qualities, while yang (bright) represents masculine, aggressive, or forceful qualities. In a human body, a vital energy of “qi” (circulating life force) flows through a complex network known as the meridian system (also referred to as “meridian network” or “channel network”) to regulate the balance of yin and yang. Insufficient, unbalanced, interrupted, or otherwise abnormal flow of qi causes imbalance of yin and yang and hence pathological conditions.

The purpose of acupuncture is to intervene at specific body sites known as “acupuncture points” (also referred to as “acupoints”) in the meridian system to adjust qi and rebalance the yin and yang. To perform acupuncture, a practitioner inserts one or more elongate metal needles percutaneously into one or more specifically selected acupoints. In many examples, multiple needles are used at a number of specific acupoints. When advancing each needle to a desired depth, the practitioner skillfully maneuvers the needle by turning, shaking, and pulsing, among other movements. Such manual maneuver (referred to as “yun zhen”) is a crucial part of an acupuncture therapy. Different abnormal conditions require different maneuvers for therapeutic effectiveness. Thus, the ability of executing such maneuvers at least partially determines the level of skill of an acupuncture therapist, and limits the number of acupuncture therapists who can acquire skills required to apply many complicated acupuncture therapies that require very precise maneuvers for safety and/or efficacy.

Having been mapped based on experience over many centuries, the meridian system and the acupoints have not been identified as anatomical features or being associated in some way to known anatomical features (such as the nervous system or circulatory system). Thus, in addition to the manual maneuver, the ability of accurately locating various acupoints also determines the level of skill of the acupuncture therapist and limits the number of acupuncture therapists who can acquire skills required to apply many complicated acupuncture therapies that require very precise locations for safety and/or efficacy.

Because acupuncture needles are made of metal, electrical currents have been introduced into tissue through the needles to stimulate the tissue to supplement, enhance, or replace the manual maneuvers, a practice known as electro-acupuncture. Examples of benefits of electro-acupuncture, or electrical stimulation using acupuncture needles, may include, but are not limited to: (1) replacing manual maneuvers of the acupuncture needles: electro-acupuncture can be applied to avoid prolonged manual maneuvering (which may become less accurate and less effective with hand fatigue), and can relieve the strict requirements of training for hand manipulation, thereby enabling a novice practitioner to achieve the same or comparable effects of needle maneuvers by an experienced practitioner; (2) providing longer stimulation duration to achieve desired therapeutic effects: electrical stimulation can be turned on for an entire therapy session, during which the practitioner can attend to other patients, thereby increasing the number of patients treated by the practitioner and/or the therapy duration available for each patient; (3) providing a stronger stimulation without causing tissue damage associated with twirling, lifting and thrusting the acupuncture needles, such as when high intensity stimulation is needed for difficult cases such as neuralgia or paralysis; and (4) facilitating the applications: controlling parameters of electrical stimulation is easier than performing the manual maneuvers of the acupuncture needles, especially with the modern electronics technology.

Such benefits are limited, however, at least in part by the lacking of scientific knowledge on acupuncture in general. For example, stimulation sites for electro-acupuncture still need to be manually located, and ability for programming parameters for electrical stimulation is limited by lacking of scientific understanding of how the human body responds to various maneuvers of acupuncture needles by the practitioner's hand.

The present subject matter provides a multi-point stimulation and data collection system for medical applications. In various embodiments, the system can use modern technologies to facilitate, assist, supplement, enhance, and/or replace various aspects of traditional acupuncture therapy. In various embodiments, the system can perform diagnostic and/or therapeutic functions using components developed by at least partially using acupunctural concepts, techniques, and/or devices. In various embodiments, the system can include a central station, one or more substations, and one or more input/output devices (IODs). The central station is remotely connected to at least one of the substations via a communication link. Each substation is locally or remotely connected to at least one IOD. Each IOD is physically and electrically connected to at least electrode of any type and shape (e.g., needle or patch). The electrode is attached onto or inserted into the skin of a patient. In various embodiments, the system can operate in a clinical environment where multi-point stimulation on or under the skin is performed for diagnosis of or therapy for certain medical conditions. Under guidance of a pre-designed study protocol, the system can collect data and deliver stimuli through electrodes. The data collection and stimulation delivery can be executed manually, automatically, or semi-automatically based on specific design of the diagnosis and/or therapy protocol. During a diagnosis and/or therapy session, the system can monitor the progress of the diagnosis and/or therapy in real-time, and can organize, classify, and store the collected data for post-study analysis.

In this document, a “user” can include a trained professional, such as a physician or other caregiver, who uses the methods and apparatuses discussed in this document to treat one or more patients. A “patient” can include any person from whom one or more signals are sensed, and/or to whom one or more therapies are delivered, using the methods and apparatuses discussed in this document. The patient may be a person seeking medical help and/or participating in a clinical study. A “person” can refer to such a patient.

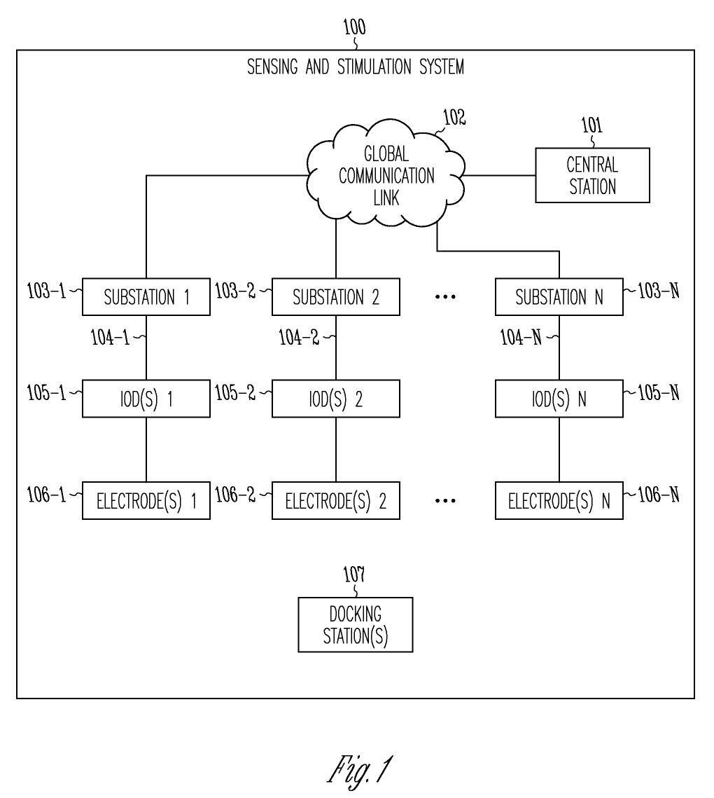

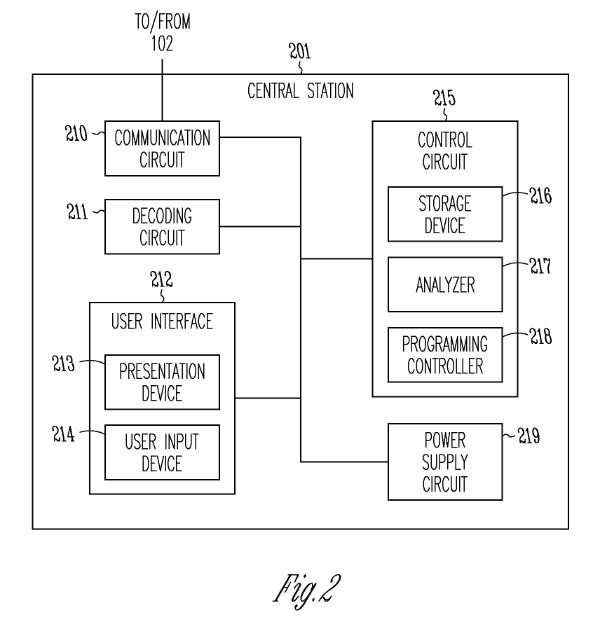

FIG. 1 is a block diagram illustrating an embodiment of a sensing and stimulation system 100. System 100 can include a central station 101, a global communication link 102, one or more substations 103, one or more local communication links 104, one or more input/output devices (IODs) 105, electrodes 106, and one or more docking stations 107. An example of central station 101 is further discussed below with reference to FIG. 2.

In various embodiments, global communication link 102 can include a wireless communication network and/or a wired communication network.

Examples of global communication link 102 include the Internet, an intranet, and a cellular network.

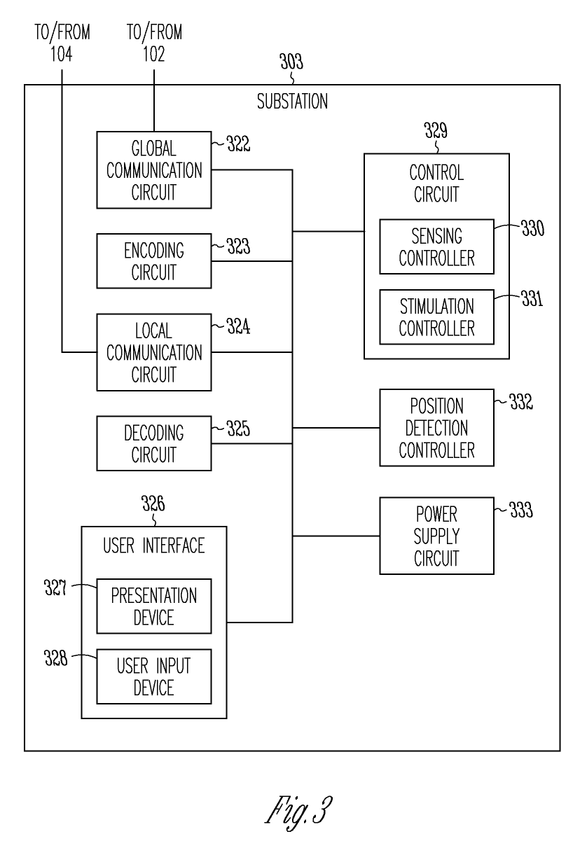

Substation(s) 103 can include one or more substations each communicatively coupled to central station 101 via global communication link 102. While a plurality of substations 103-1, 103-2, . . . and 103-N are illustrated in FIG. 1, various embodiments can include any number of substations, subjected to the capability of central station 101 (i.e., N=1, 2, 3, . . . ). An example for one of substation(s) 103 is further discussed below with reference to FIG. 3.

Local communication link(s) 104 can each include a wired communication link or a wireless communication link coupled between one of substation(s) 103 and one of IOD(s) 105. Examples of the wired communication link include a cable such as a USB cable or Ethernet cable. Examples of the wireless communication link includes a Bluetooth® link, a Bluetooth low energy (BLE) link, an IEEE 802.11 (wireless LANs) link, an 802.15 (WPANs) link, an 802.16 (WiMAX) link, an electromagnetic communication link (e.g., an RF far-field or inductive link), an optical communication link (e.g., an infrared link), an acoustic communication link (such as an ultrasound link) and a cellular communication link (e.g., CDMA, GSM, ZigBee, or Ultra-wideband (UWB) link).

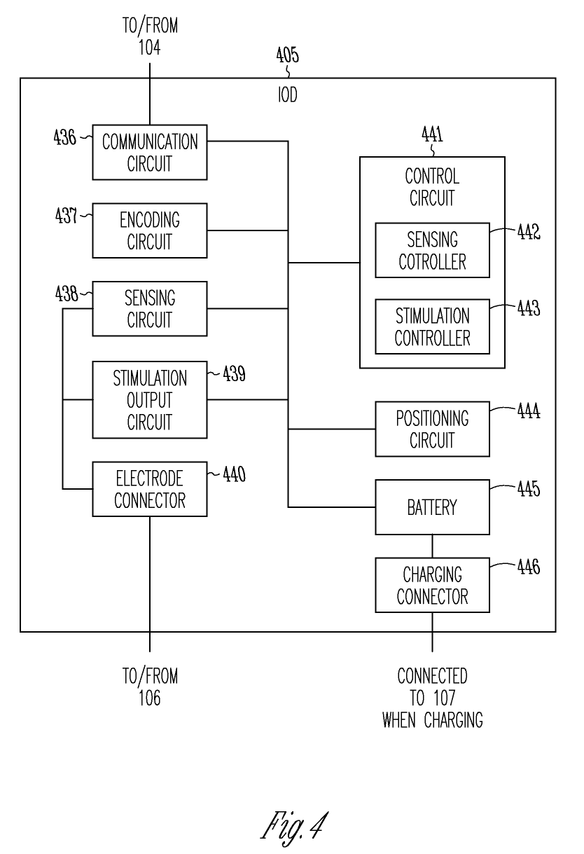

IOD(s) 105 can each be communicatively coupled to one of substation(s) 103 via one of local communication link(s) 104. In FIG. 1, each of IOD(s) 105-1, 105-2, . . . and 105-N represent a set of one or more IODs communicatively coupled to one of substations 103-1, 103-2, . . . and 103-N via one of more local communication links 104-1, 104-2, . . . and 104-N, respectively. An example for one of IOD(s) 105 is further discussed below with reference to FIG. 4.

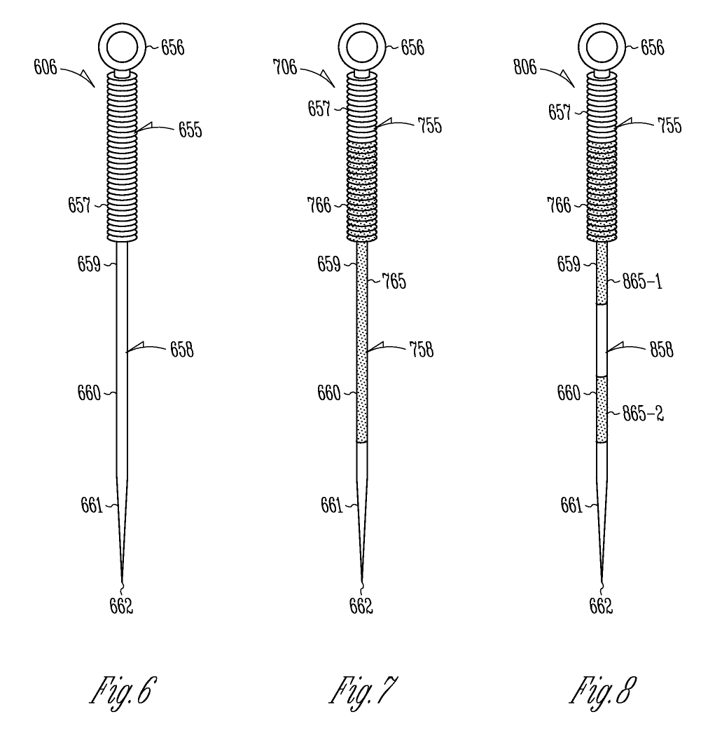

Electrodes 106 provide for an interface between system 100 and one or more patients being diagnosed, monitored, and/or treated using system 100. In FIG. 1, each of electrode(s) 106-1, 106-2, . . . and 106-N represent a set of one or more electrodes electrically connected to IOD(s) 105-1, 105-2, . . . and 105-N, respectively. Each IOD functions as a front-end device that is connected to a patient via one or more electrodes. Examples of electrodes 106 can include surface electrodes (e.g., skin patch electrodes, transcutaneous electrical nerve stimulation (TENS) electrodes, and surface neuromuscular sensing electrodes) and percutaneous electrodes (e.g., needle electrodes). Examples of needle electrodes are further discussed below with reference to FIGS. 6-14.

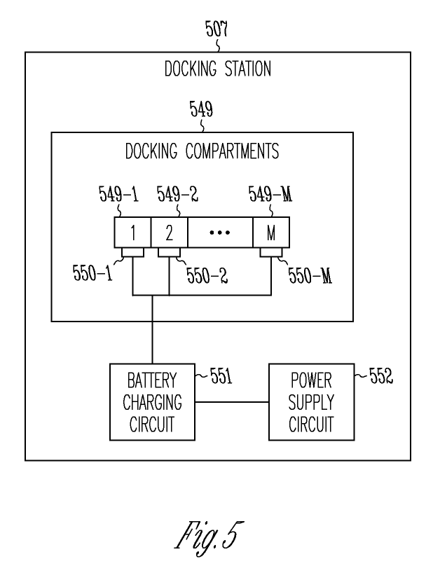

Docking station(s) 107 each allow one or more IODs 105 to dock and recharged. An example for one of docking station(s) 107 is further discussed below with reference to FIG. 5.

In various embodiments, circuits of system 100, including its various embodiments discussed in this document, may be implemented using a combination of hardware and software. For example, portions of various communication circuits, encoding and decoding circuits, circuits of user interfaces, positioning circuits, and control circuits, as discussed below in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 2 is a block diagram illustrating an embodiment of a central station 201, which represents an example of central station 101. Central station 201 can include a communication circuit 210, a decoding circuit 211, a user interface 212, a control circuit 215, and a power supply circuit 219. In various embodiments, central station 201 can be implemented on a generic computer and/or as a custom-made device.

Communication circuit 210 can communicate with each substation of substation(s) 103 via global communication link 102. In various embodiments, communication circuit 210 can receive data from each substation of substation(s) 103. The data can be acquired by the substation and include data indicative of operation status of the substation and operation status of each IOD of IOD(s) 105 communicatively coupled to that substation. In one embodiment, the data can be received by communication circuit 210 in real time. In some embodiments, central station 201 only monitors substation(s) 103 and perform analyses of the data acquired by substation(s) 103, such as for statistical studies. In other embodiments, central station 201 can also be used to control the operation of substation(s) 103 and/or IOD(s) 105.

Decoding circuit 211 can identify each substation of substation(s) 103 that is connected to central station 201 via global communication link 102 using a unique substation identification code assigned to that substation. In various embodiments, the identification code can indicate the owner or user, the patient, the location, etc., associated with the substation identified by the identification code.

User interface 212 can include a presentation device 213 to present information received from each substation and a user input device 214 to receive commands and other information from the user. In various embodiments, user interface 212 can include a graphic user interface (GUI). In various embodiments, portions of presentation device 213 and user input device 214 can be integrated into an interactive touchscreen. In various embodiments, presentation device 213 can include any type of presentation device, such as interactive or non-interactive screens. Use input device 214 can include any type of user input devices, such as touchscreen, keyboard, keypad, touchpad, trackball, joystick, and mouse.

Control circuit 215 controls operation of central station 201. Control circuit 215 can include a storage device 216, an analyzer 217, and a programming controller 218. Storage device 216 can store data received from substation(s) 103 and results of analysis performed by central station 201. Analyzer 217 analyzes the data received from each substation of substation(s) 103. In various embodiments, the analysis performed by analyzer 217 can include detection of specified type information from the data received from each substation. Examples of such specified type information include the number of IOD(s) 105 communicatively coupled to the substation, a current operation mode of each IOD communicatively coupled to the substation (e.g., a sensing mode and a stimulation mode), an operation status of each IOD communicatively coupled to the substation (e.g., normal function and abnormal function), and/or the battery levels of the substation and/or each IOD communicatively coupled to the substation. In various embodiments, analyzer 217 can generate one or more reports based on the analysis. In various embodiments, analyzer 217 can organize and/or classify the reports, selectively store one or more of the reports in storage device 216, and/or selectively present one or more of the reports using presentation device 213. In various embodiments, analyzer 217 can organize and/or classify the reports, selectively store one or more of the reports in storage device 216, and/or selectively present one or more of the reports using presentation device 213 according to specified criteria. The specified criteria can include predetermined criteria and/or user-selected criteria received using user input device 214. Programming controller 218 allows the user to adjust the operation of central station 201 and/or various other portions of system 100, such as using user interface 212. In embodiments in which central station 201 can be used to control the operation of substation(s) 103 and/or IOD(s) 105, programming controller 218 can generate programming codes to be transmitted to one or more of substation(s) 103 and/or IOD(s) 105 to be programmed or reprogrammed via global communication link 102. Programming controller 218 can generate programming codes based on predetermined programming instructions stored in storage device 216, user commands received using user input device 214, outcome of the analysis performed by analyzer 217, the operation status of each of substation(s) 103, and/or the operation status of each of IOD(s) 105. Power supply circuit 219 provides power for the operation of central station 201.

FIG. 3 is a block diagram illustrating an embodiment of a substation 303, which represents an example of one of substation(s) 103. Substation 303 can include a global communication circuit 322, an encoding circuit 323, a local communication circuit 324, a decoding circuit 325, a user interface 326, a control circuit 329, a position detection controller 332, and a power supply circuit 333. In various embodiments, substation 303 can be implemented on a generic computer and/or as a custom-made device.

Global communication circuit 322 can communicate with central station 101 via global communication link 102, including transmitting the data acquired by substation 303 to central station 101. Encoding circuit 323 can generate the unique substation identification code for substation 303. Local communication circuit 324 can communicate with one or more IODs of IOD(s) 105 via one or more local communication links of local communication link(s) 104. In various embodiments, local communication circuit 324 can receive signals from each IOD connected to substation 303 via a link of local communication link(s) 104. Examples of such received signals include one or more physiological signals acquired by the IOD (e.g., a signal indicative of electrical activities in the patient, a signal indicative of a temperature in the patient, and/or a signal indicative of a chemical parameter measured from the patient, such as pH value), one or more status signals indicative of operational status of the IOD (e.g., a current operation mode of the IOD, normal function of the IOD, and/or abnormal function of the IOD), a unique IOD identification code associated with the IOD indicating that IOD is connected, and/or a signal indicative of a strength and/or data transmission quality of the local communication link coupled between the IOD and substation 303. In various embodiments, local communication circuit 324 can also transmit signals to each IOD connected to substation 303 via a link of local communication link(s) 104. Examples of such transmitted signals include signals for controlling operation of the IOD (e.g., sensing parameters and stimulation parameters). Decoding circuit 325 can identify each IOD connected to substation 303 via a link of local communication link(s) 104 by a unique IOD identification code associated with that IOD.

User interface 326 can include a presentation device 327 to present information received from central station 101 and IOD(s) 105 and a user input device 328 to receive commands and other information from the user. In various embodiments, user interface 326 can include a graphic user interface (GUI). In various embodiments, portions of presentation device 327 and user input device 328 can be integrated into an interactive touchscreen. In various embodiments, presentation device 327 can include any type of presentation device, such as interactive or non-interactive screens. Use input device 328 can include any type of user input devices, such as touchscreen, keyboard, keypad, touchpad, trackball, joystick, and mouse. In various embodiments, presentation device 327 can present a graphic or non-graphic representation of a target map. The target map can include, for example, the meridian system including meridians (routes), acupoints, trigger points, neuro-acupuncture points, and/or other specified electrostimulation points on the skin/body surface (cutaneous sites) and/or underneath the skin (subcutaneous sites), location of each electrode of electrodes 106 currently deployed (connected to each IOD of IOD(s) 105 that is connected to substation 303 via a link of local communication link(s) 104), and/or status of each IOD of IOD(s) 105 that is connected to substation 303 via a link of local communication link(s) 104.

Control circuit 329 controls operation of substation 303 and can include a sensing controller 330 and a stimulation controller 331. Sensing controller 330 can process the signals received from each IOD that is connected to substation 303. In various embodiments, sensing controller 330 can generate sensing parameters for controlling sensing functions of the IOD. In various embodiments, sensing controller 330 can analyze the processed signals including organizing the processed signals and classifying the processed signals, selectively store one or more of processed signals in substation 303, selectively present one or more of the processed signals using presentation device 327, and/or selectively transmit one or more of processed signals to central station 101. In various embodiments, sensing controller 330 selective stores one or more of processed signals in substation 303, selectively presents one or more of processed signals using presentation device 327, and/or selectively transmits one or more of processed signals to central station 101 according to specified criteria. The specified criteria can include predetermined criteria and/or criteria received from the user using user input device 328. In various embodiments, sensing controller 330 can detect location of each deployed electrode of electrodes 106 that is connected to substation 303 via an IOD of IOD(s) 105 and a link of local communication link(s) 104 on the target map. In one embodiment, sensing controller 330 receives the location of each electrode manually entered by the user using user input device 328. In another embodiment, sensing controller 330 detects the location of each electrode automatically using positioning detection controller 332, as further discussed below. In various embodiments, sensing controller 330 can detect characteristics of each deployed electrode including, for example, its type, mechanical parameters, and electrical parameters. In one embodiment, sensing controller 330 receives such characteristics from the user manually entering them using user input device 328. In various embodiments, sensing controller 330 can detect a current operation mode of each IOD that is connected to substation 303. Examples of the current operation mode can include standby, sensing, and/or stimulation. In various embodiments, sensing controller 330 can detect a battery level of each IOD that is connected to substation 303. In various embodiments, sensing controller 330 can identify signals sensed by each deployed electrode that is connected to substation 303. In various embodiments, sensing controller 330 can generate sensing parameters instructing one or more IODs connected to substation 303 to sense one or more specified type signals using one or more specified electrodes. In one embodiment, sensing controller 330 generates such sensing parameters after delivery of a stimulus or after a delay period following the delivery of the stimulus.

Stimulation controller 331 can generate stimulation parameters controlling delivery of stimulation from each IOD connected to substation 303. In various embodiments, the stimulation parameters can define a stimulus as a single pulse, a burst of pulses, a sequence of bursts, a continuous waveform, etc. The stimulation parameters can specify a constant or varying (e.g., modulated) pulse frequency and/or a constant or varying (e.g., modulated) pulse amplitude. In various embodiments, stimulation controller 331 can generate the stimulation parameters using the signals processed by sensing controller 330 and/or outcome of the analysis of processed signals performed by sensing controller 330. In various embodiments, stimulation controller 331 can instruct each IOD connected to substation 303 to deliver a stimulus using the outcome of the analysis of processed signals performed by sensing controller 330 and one or more predetermined thresholds. In one embodiment, stimulation controller 331 generates the stimulus and transmits the stimulus to an intended IOD for delivery through one or more specified electrodes connected to the IOD (i.e., the IOD simply relays the stimuli). In another embodiment, stimulation controller 331 generates stimulation parameters and transmits the stimulation parameters to the IOD, which then generates the stimulus and delivers the stimulus through one or more specified electrodes connected to the IOD according to the stimulation parameters.

In various embodiments, control circuit 329 can generate a report for each diagnostic and/or therapeutic session with the patient. The report can include, for example, the patient's demographics (e.g., age and gender), identification and location of each IOD connected to substation 330 on the target map, parameters of the stimuli delivered during the session (e.g., type and duration), and/or notes received from the user using user input device 328 (e.g., observations and comments regarding effects of the delivery of the stimuli).

Position detection controller 332 allows substation 303 to detect location of each IOD connected to substation 303 using triangulation. When a deployed electrode connected to each IOD is directly attached to that IOD or placed in close proximity of that IOD, it allows for detection of approximate location of the deployed electrodes. Automatic detection of IOD locations using triangulation is possible with three or more IODs (referred to as pinning IODs) each having a positioning circuit (as discussed below with reference to FIG. 4) are placed at certain selected areas on the patent and emit the acoustic signals. The time of the emission is controlled by position detection controller 332 and synchronized with the IODs connected to substation 303, such as by transmitting a synchronization mark to each of the connected IODs. In response to instructions of position detection controller 332, each of the connected IODs receives the acoustic signal emitted from the pinning IODs, and measures the time delay between the emission of the acoustic signal from pinning IODs and the reception of the acoustic signal by the IOD. The time delay is reported back to sensing controller 330 and used by position detection controller 332 to determine the location of each IOD relative to the known locations of the pinning IODs using the triangulation method.

Power supply circuit 333 can provide power for the operation of substation 303. In various embodiments, power supply circuit 333 can include a battery, an AC adaptor, or both.

FIG. 4 is a block diagram illustrating an embodiment of an IOD 405, which represents an example of one of IOD(s) 105. IOD 405 can include a communication circuit 436, an encoding circuit 437, a sensing circuit 438, a stimulation output circuit 439, an electrode connector 440, a control circuit 441, a positional circuit 444, a battery 445, and a charging connector 446. In various embodiments, IOD 405 can include a chassis housing communication circuit 436, encoding circuit 437, sensing circuit 438, stimulation output circuit 439, control circuit 441, positional circuit 444, and battery 445, while electrode connector 440 and charging connector can be incorporated onto the chassis. In various embodiments, IOD 405 is visually identifiable using an identification feature incorporated onto the chassis. In one embodiment, the unique IOD identification code is visibly labeled or displayed on the IOD chassis.

Communication circuit 436 can communicate with a substation of substation(s) 103 that is connected to IOD 405 via a link the local communication link(s) 104. In various embodiments, communication circuit can receive signals transmitted from the connected substation (e.g., the stimulus or stimulation parameters and/or the sensing parameters) and/or transmit signals to the connected substation (e.g., one or more physiological signals sensed by sensing circuit 438, the one or more status signals indicative of operational status of IOD 405, and/or the unique IOD identification code associated with IOD 405). Encoding circuit 437 can generate the unique IOD identification code for IOD 405.

Sensing circuit 438 can sense one or more physiological signals through one or more electrodes of electrodes 106 that are connected to IOD 405 through electrode connector 440. In various embodiments, sensing circuit 438 can sense one or more physiological signals according to the sensing parameters. Examples of the one or more physiological signals can include neuromuscular signals, signals allowing for characterization of electrical properties of the meridian system, a signal indicative of temperature (through an electrode with an embedded temperature sensor), and/or a signal indicative of pH value and/or other chemical parameters (through an electrode with an embedded chemical sensor).

Stimulation output circuit 439 can deliver the stimuli through one or more electrodes of electrodes 106 that are connected to IOD 405 through electrode connector 440. In various embodiments, the stimuli can include electrical current, such as in the form of pulses. In various embodiments, the stimuli can also include various forms of magnetic, thermal, acoustic, optical, and mechanical energy as well as chemical and biological agents.

Electrode connector 440 allow for physical and electrical connections to the one or more electrodes, directly or through one or more cables or leads. In various embodiments, electrode connector 440 can provide one or more electrical connections between one or more electrodes and sensing circuit 438 and/or one or more electrical connections between one or more electrodes and stimulation output circuit 439. In one embodiment, the one or more electrical connections between one or more electrodes and sensing circuit 438 and the one or more electrical connections between one or more electrodes and stimulation output circuit 439 include one or more common electrical connections.

Control circuit 441 controls operation of IOD 405 and can include a sensing controller 442 and a stimulation controller 443. Sensing controller 442 can control the sensing of the one or more physiological signals using one or more predetermined sensing parameters and/or one or more sensing parameters received from the connected substation. Stimulation controller 443 can control the delivery of the stimuli using the stimulus received from the connected substation or the stimulation parameters received from the connected substation.

Positioning circuit 444 allows for determination of position of IOD 405 relative to one or more known locations on the patient's body using triangulation, and can include one or more acoustic transducers to emit and receive acoustic signals, such as audible or ultrasonic signals. Positioning circuit 444 can receive commands from substation 303 to emit or receive acoustic signals, calculate a time interval between emission of the acoustic signal from another IOD (i.e., a pinning IOD) and the reception of that acoustic signal by IOD 405, and transmit the time interval to the connected substation 303.

Battery 445 can provide energy for operation of IOD 405. In various embodiments, battery 445 includes a rechargeable battery that can be recharged by electrically connecting to a docking station of docking station(s) 107 through charging connector 446. In various embodiment, control circuit 441 can perform battery management functions including detecting a battery status (e.g., an energy level) of battery 445 and transmit the battery status to the connected substation.

FIG. 5 is a block diagram illustrating an embodiment of a docking station 507, which represents an example of one of docking station(s) 107. Docking station 507 can include one or more docking compartments 549 each accommodate an IOD of IOD(s) 105, a battery charge circuit 551, and a power supply circuit 552. In various embodiments, docking compartments 549-1, 549-2, . . . 549-M (M=1, 2, . . . ) can each include a visible identification feature that identifies an IOD of IOD(s) 105 that is intended to be docked in the docking compartment. In one embodiment, docking compartments 549-1, 549-2, . . . and 549-M are each labeled with the unique IOD identification code of the IOD intended to be docked. Docking compartments 549-1, 549-2, . . . and 549-M each have a structure to mate with the IOD chassis of the IOD intended to be docked to securely hold the IOD in place, and include battery charging connectors 550-1, 550-2, . . . and 550-M each connected to battery charging circuit 551 and configured to mate with the charging connector (e.g., charging connector 446) of the docked IOD. Power supply circuit 552 can supply power for operation of docking station 507. Battery charging circuit 551 can receive power from power supply circuit 552 and convert the received power to a form suitable for charging the docked IOD(s).

In an example of application, system 100 works in a clinical environment where surface stimulation and/or percutaneous stimulation are performed. Guided by a pre-designed protocol, the user turns on the power of a substation. After an initialization of the substation (including communication to the connected central station), the user decides where to place electrodes, on the skin or in the tissue under the skin, at various locations based on a specific therapy methodology (e.g., acupuncture therapy, trigger-point therapy, or neurostimulation therapy). Once decided, the user marks the location of each electrode onto a target map (showing the meridian system for example). Alternatively, the substation can localize the electrodes automatically using triangulation. Then, the user places each electrode on the skin or in the tissue according to the decided locations. After turning on the IODs, there is a period of initialization when the substation establishes communication with each IOD. When the therapy session begins, the user enters various commands from the substation to instruct one or more IODs to either record data or deliver specific stimulus according to the pre-designed protocol. The protocol can also be executed by the substation when sensing and stimulation can be controlled automatically. During the therapy session, the user can monitor the progress including the status of each IOD in real-time, and adjust the procedure whenever necessary according to the protocol.

Various embodiments of system 100, central station 201, substation 303, IOD 405, and docking station 507 may not include all the components illustrated in FIGS. 1, 2, 3, 4, and 5, respectively, and may depend on the desired functionality of each system or device for each specific application. For example, position detection controller 332 of substation 303 and positioning circuit 444 of IOD 405 are not needed if automatic detection of IOD/electrode locations using triangulation is not performed, and components providing system 100 with sensing capabilities may not be needed if the system is used in a stimulation-only electro-acupuncture application.

### Examples of Electrodes

FIGS. 6-14 illustrate various embodiments of needle electrodes and their use in a medical diagnostic and/or therapeutic system, such as their use as electrode(s) 106 in system 100. The needle electrodes and their accessories, including sizes and shapes of their various features, are illustrated FIGS. 6-14 by way of example, but not by way of restriction.

FIG. 6 is an illustration of an embodiment of an acupuncture needle 606. Acupuncture needle 606 represents an example of an electrode such as one of electrode(s) 106, and includes a handle 655 and a needle body 658 coupled to handle 655. In various embodiments, needle body 658 can be substantially bendable and has sufficient stiffness for percutaneous insertion into tissue of the patient by applying force to handle 655. In some embodiments, both handle 655 and needle body 658 are substantially bendable.

In various embodiments, handle 655 includes a connector portion electrically coupled to needle body 658 to allow for an electrical connection between needle body 658 and a medical device such as one of IOD(s) 105. In the illustrated embodiment, handle 655 includes a ring top 656 and a handle shaft 657. Handle shaft 657 is coupled between needle body 658 and ring top 656. In various embodiments, handle shaft 657 can include a coiled structure or any other structure suitable for being held by a hand to perform acupuncture maneuvering. Portions of ring top 656 and/or handle shaft 657, wherever not electrically insulated, can be used as the connector portion. In other embodiments, handle 655 can include a connector specifically configured to allow for the electrical connection using a cable or a connector specifically configured to be directly attached to the medical device such as one of IOD(s) 105. In various embodiments, handle 655 can include an identification feature, such as a color. For example, when a plurality of acupuncture needles are used in the same system or for the same patient, their handles can be color codes for identifying different types and/or sizes.

Needle body 658 includes a proximal end portion 659 coupled to handle 655, a distal end portion 661 including a needle tip 662, and an elongate body shaft 660 coupled between proximal end portion 659 and distal end portion 661. Needle tip 662 is shaped and sized to pierce tissue of the patient without causing excessive pain to the patient or breakage of acupuncture needle 606. In various embodiments, the length of needle body 658 (which may be referred to as the length of the acupuncture needle) can be in the range of approximately 13-130 mm, and the diameter of elongate body shaft 660 (which may be referred to as the diameter of the acupuncture needle) is in the range of approximately 0.16-0.46 mm. Acupuncture needle 606 can be made of a metal material such as stainless steel. In some embodiments, acupuncture needle 606 can be gold or silver coated. In some embodiments, handle 655 and needle body 658 can be formed using a single piece of metal wire.

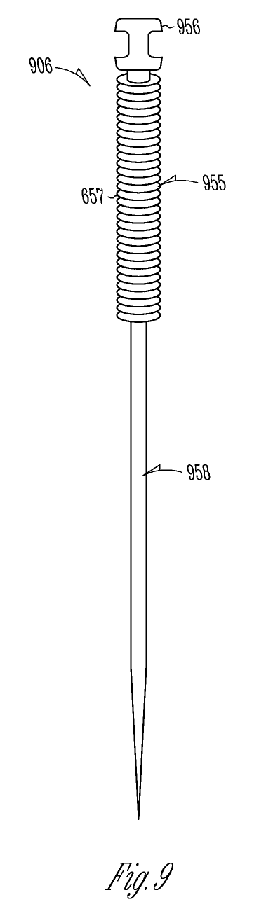

While acupuncture needles are specifically discussed as examples of needle electrodes, various features incorporated into an acupuncture needle, as discussed in this document such as with reference to FIGS. 7-9, 11, and 13, can also be incorporated into other types of needles for use as electrodes such as electrode(s) 106 in system 100. Examples of such other types of needles include biopsy needles and injection needles. In some embodiments, one or more sensors can be embedded in the needle body of a needle electrode, such as a temperature sensor and a chemical sensor.

FIG. 7 is an illustration of an embodiment of a partially insulated acupuncture needle 706, which represents another example of an electrode such as one of electrode(s) 106. Acupuncture needle 706 is substantially identical or similar to acupuncture needle 606 but is partially insulated. In the illustrated embodiment, acupuncture needle 706 includes a handle 755 with an insulation layer 766 and a needle body 758 with an insulation layer 765. In various embodiments, handle 755 can include no insulation layer 766, insulation layer 766 covering about the whole length of handle shaft 657, or insulation layer 766 covering any portion of handle shaft 657. Insulation layer 765 can cover any portion of needle body 758. In various embodiments, insulation layer 765 can have a length in a range of approximately ⅔ to ⅘ of the length of needle body 758, and can have a thickness in a range of approximately 3-200 μm.

In various embodiments, insulation layer 765 and insulation layer 766 can be formed by coating. In one embodiment, insulation layer 765 and insulation layer 766 are formed in the same coating process. In other words, insulation layer 765 extends to a portion of handle 766. The coating can be applied using a standard industry procedure. For example, the needles are first cleaned with alcohol and then put on low energy plasma to improve adhesion. After masking the area that is not to be coated, a thin layer of the coating is applied via chemical vapor deposition at room temperature. In various embodiments, insulation layer 765 is formed by coating using an electrically non-conductive material that is non-toxic and biocompatible. The resulting insulation layer 765 can sustain acupunctural maneuvers and a sterilization procedure. Examples of such electrically non-conductive material can include electrically non-conductive polymers, such as Parylene (by Diamond-MT, Johnstown, Pa.). In various embodiments, insulation layer 765 is formed by coating using a thermally non-conductive material that is non-toxic and biocompatible. The resulting insulation layer 765 can sustain acupunctural maneuvers and a sterilization procedure. Examples of such thermally non-conductive material can include polyacrylonitrile (PAN) and polyamide. In various embodiments, insulation layer 765 is formed by coating using a diffusible chemical material that is non-toxic and biocompatible. The resulting insulation layer 765 can sustain acupunctural maneuvers and a sterilization procedure. Examples of such thermally non-conductive material can include drugs, such as antibiotic and/or anti-inflammatory agents.

In various embodiments, insulation layer 765 can be formed by multi-layer coating using different coating materials. For example, insulation layer 765 can be formed by (1) coating the electrically non-conductive material over needle body 661 and then coating the thermally non-conductive material over the electrically non-conductive material, (2) coating the thermally non-conductive material over needle body 661 and then coating the electrically non-conductive material over the thermally non-conductive material, (3) coating the electrically non-conductive material over needle body 661 and then coating the diffusible chemical material over the electrically non-conductive material, (4) coating the thermally non-conductive material over needle body 661 and then coating the diffusible chemical material over the thermally non-conductive material, (5) coating the electrically non-conductive material over needle body 661, coating the thermally non-conductive material over the electrically non-conductive material, and then coating the diffusible chemical material over the thermally non-conductive material, and (6) coating the thermally non-conductive material over needle body 661, coating the electrically non-conductive material over the thermally non-conductive material and then coating the diffusible chemical material over the electrically non-conductive material. In various embodiments, such multi-layer coating can be applied to form both insulation layer 765 and insulation layer 766.

FIG. 8 is an illustration of another embodiment of a partially insulated acupuncture needle 806, which represents another example of an electrode such as one of electrode(s) 106. Acupuncture needle 806 is substantially identical or similar to acupuncture needle 706, but include a needle body 858 with two segments covered by insulation layers 865-1 and 865-2. Insulation layers 865-1 and 865-2 can be formed using the same coating process that is used to form insulation layer 765. In various embodiments, layers 865-1 and 865-2 can have a total length in a range of approximately ⅔ to ⅘ of the length of needle body 758, and can each have a thickness in a range of approximately 3-200 μm.

While acupuncture needles 706 and 806 are illustrated as examples, in various embodiments, a partially insulated acupuncture needle according to the present subject matter can include one segment of the insulation layer over a substantial portion of the needle body, or a plurality of segments of the insulation layer over a substantial portion of the needle body with each segment of the insulation layer separated from one or more other segments of the insulation layer by an non-insulated portion of the needle body. The insulation layer with one or more segments can have a total length in a range of approximately ⅔ to ⅘ of the length of the needle body.

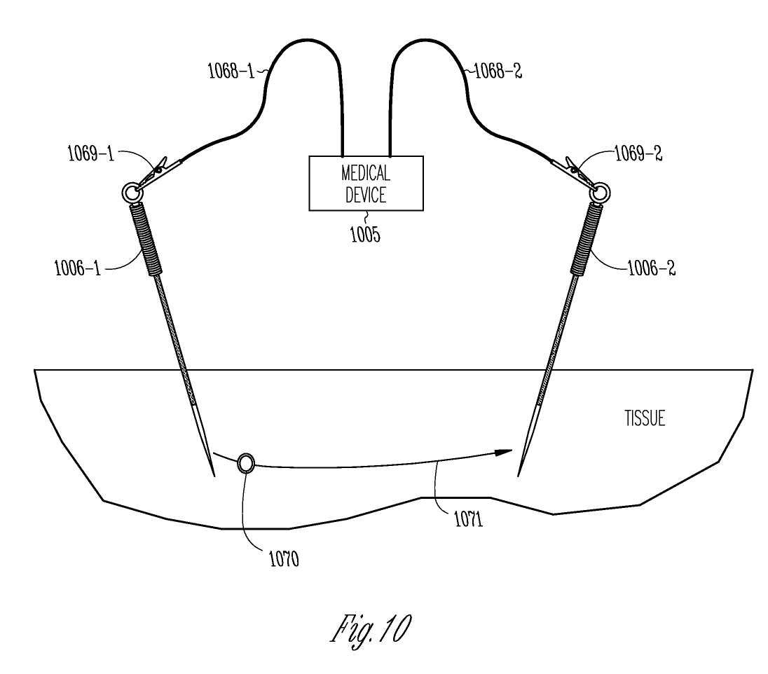

FIG. 9 is an illustration of an embodiment of an acupuncture needle 906, which represents another example of an electrode such as one of electrode(s) 106. Acupuncture needle 906 includes a handle 955 and a needle body 958. Handle 955 includes an electrical connector 956 that is custom shaped and sized to mate with a custom shaped and sized electrode connector. In one embodiment, the electrical connector 956 that is custom shaped and sized to be attached to a medical device such as IOD 405 via electrode connector 440, which can be custom shaped and sized to mate with electrical connector 956. In various embodiments, needle body 958 can be substantially identical or similar to needle body 658, 758, or 858. That is, needle body 958 can be non-insulated or partially insulated with one or more segments of insulation layer.

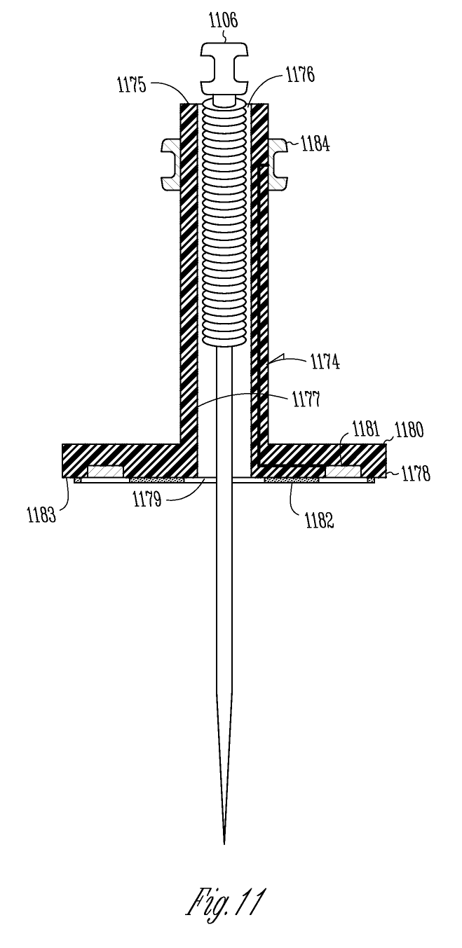

FIG. 10 is an illustration of an embodiment of acupuncture needles 1006 connected to a medical device 1005, such as an IOD of IOD(s) 105, and inserted in tissue. Acupuncture needles 1006-1 and 1006-2 can each be one of acupuncture needle 606, 706, 806, or 906, and electrically connected to medical device 1005 via an electrode connector 1069-1 with a cable 1068-1 and an electrode connector 1069-2 with a cable 1068-2, respectively.

In the illustrated embodiment, acupuncture needle 1006-1 is inserted into the tissue with the non-insulated portion of its needle body placed adjacent an intended target 1070 for sensing from and/or delivering stimulation to that target. When compared to uninsulated acupuncture needles, use of two partially insulated acupuncture needles 1006-1 and 1006-2 allows a current 1071 flowing between them to be more focused on intended target 1070, thereby providing for a better signal-to-noise ratio for sensing, a better energy efficiency for stimulation, and/or reduced side effects related to stimulation of unintended areas in the tissue. This can significantly reduce the risk to the patient when delivering stimulation to sites in sensitive areas of the patient's body. For example, when such sites are near the patient's heart, a current spread to unintended areas may cause the heart to beat irregularly, and when such sites are in or near the spinal cord, a current spread to unintended areas may cause nerve fibers in or around the spinal cord to respond erroneously. Thus, the use of partially insulated acupuncture needles may reduce the risk of performing electro-acupuncture in acupoints that are known to be risky if not accurately targeted.

Various embodiments can use a variety of configurations for electro-acupuncture that employ one or more partially-insulated needles as discussed in this document. Such configurations can use partially-insulated needles with same or different coating patterns, or partially-insulated and non-insulated acupuncture needles. It is desirable to place a partially-insulated acupuncture needle with its non-insulated portion adjacent the target site. Thus, in various embodiments, partially-insulated acupuncture needles with different sizes and different coating patterns can be provided to allow the user to choose an approximately optimal acupuncture needle for the target site of a certain depth under the skin. In various embodiments, the sensing capability of system 100 may facilitate such identification of the approximately optimal acupuncture needle.

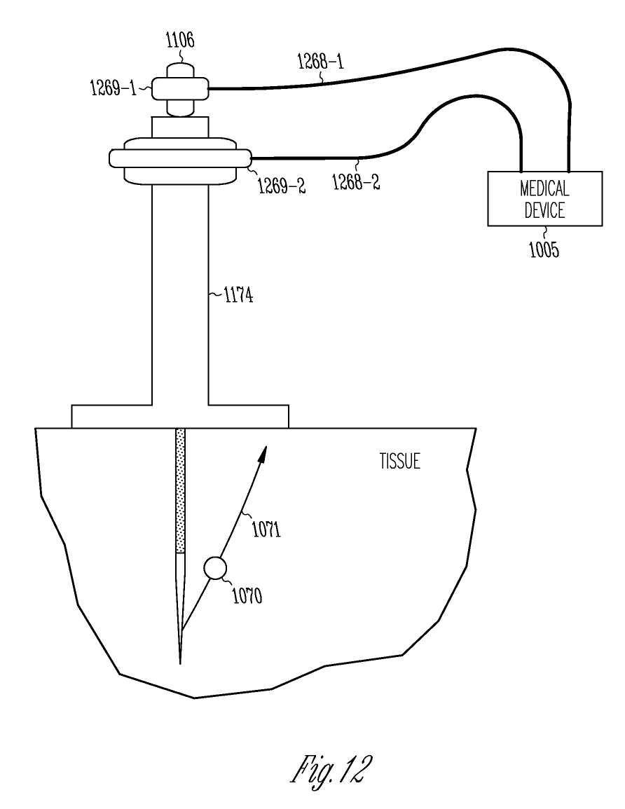

FIG. 11 is an illustration of an embodiment of an acupuncture needle 1106 and a guiding tube 1174. Acupuncture needle 1106 can represent any one of acupuncture needles 606, 706, 806, and 906. Guiding tube 1174 (shown in a cross-sectional view) can be used to assist insertion of acupuncture needle 1106 into tissue of the patient, and can function as a needle stopper that stops further insertion of acupuncture needle 1106 when its handle is entirely or substantially inside guiding tube 1174.

In various embodiments, guiding tube 1174 can be an elongate tube made of an electrically non-conductive material that is substantially stiff. The elongate tube includes a proximal end 1175 including a proximal opening 1176, a distal end 1178 including a distal opening 1179, and a lumen 1177 between proximal opening 1176 and distal opening 1179 to allow the needle tip of acupuncture needle 1106 to enter proximal opening 1176 and exit from distal opening 1179. Distal end 1178 is to contact the skin in a site where acupuncture needle 1106 is to be inserted into the patient's tissue. In various embodiments, an electrode is incorporated into distal end 1178. In the illustrated embodiment, distal end 1178 includes a disk 1180 that includes a skin-contact surface 1183, and a ring-shaped electrode 1181 is incorporated onto skin-contact surface 1183. In one embodiment, an adhesive material 1182 is applied to skin-contact surface 1183, such as adjacent to electrode 1181, to allow disk 1180 to be stably attached onto the skin. In various embodiments, disk 1180 can include one or more electrodes of any suitable shape(s) incorporated onto skin-contact surface 1183. Guiding tube 1174 includes an electrical connector 1184 that is electrically connected to electrode 1181 and allow for electrical connection to one of IOD(s) 105 directly or via a cable.

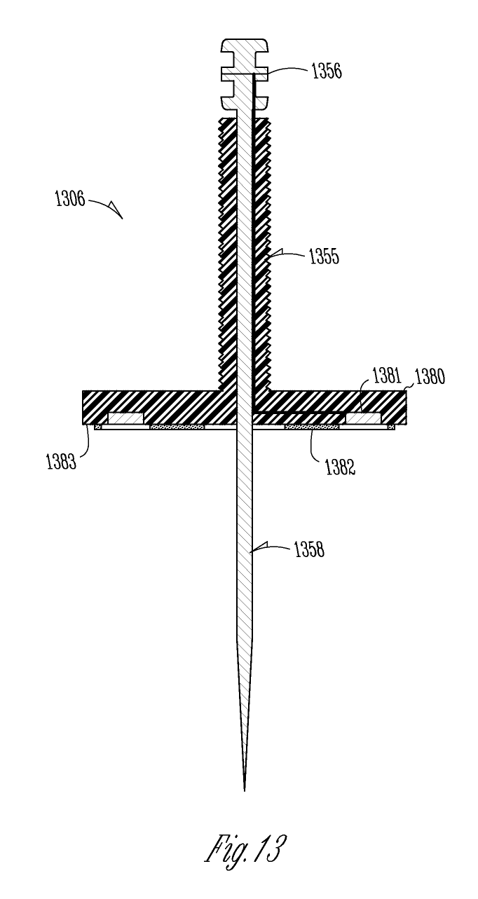

FIG. 12 is an illustration of an embodiment of acupuncture needle 1106 and guiding tube 1174 connected to medical device 1105 and inserted into the patient's tissue. The needle body of acupuncture needle 1106 and electrode 1181 of guiding tube 1174 are electrically connected to medical device 1005 via an electrode connector 1269-1 with a cable 1268-1 and an electrode connector 1269-2 with a cable 1268-2, respectively. As illustrated in FIG. 12, acupuncture needle 1106 and guiding tube 1174 provides a pair of electrodes needed to sense a signal from and/or deliver stimulation to target 1070, thereby eliminating the need for another acupuncture needle. Using a partially insulated acupuncture needle for acupuncture needle 1106 can provide the same advantages over using a non-insulated acupuncture needle as discussed above with reference to FIG. 10.

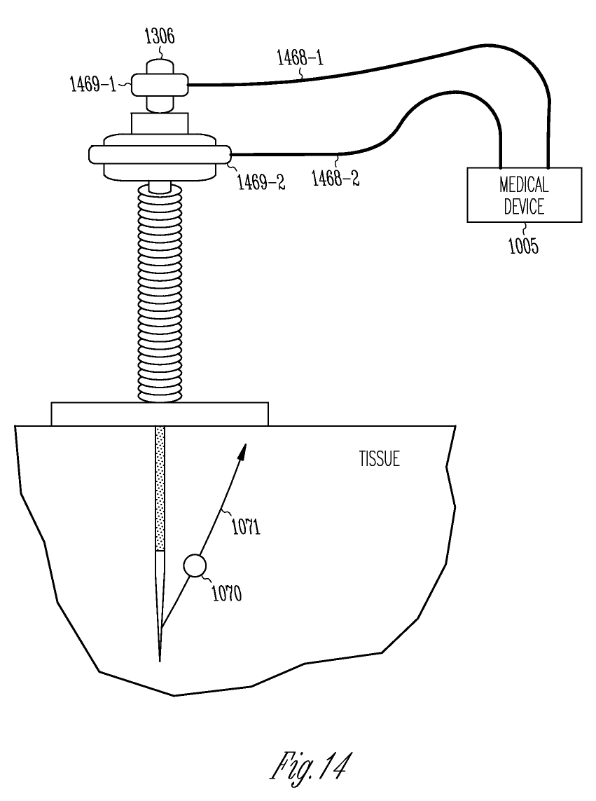

FIG. 13 is an illustration of an embodiment of an acupuncture needle 1306 (shown in cross-sectional view) that includes a handle 1355 and a needle body 1358. In various embodiments, needle body 1358 can be substantially identical or similar to needle body 658, 758, or 858. That is, needle body 1358 can be non-insulated or partially insulated with one or more segments of insulation layer. Acupuncture needle 1306 includes a disk 1380 coupled between handle 1355 and needle body 1358. In various embodiments, disk 1380 can be made of an electrically non-conductive material, and can be used to prevent needle body 1358 from over insertion into the patient's tissue. That is, disk 1380 can function as a needle stopper to stop advance of the needle tip of needle body 1358 in the tissue such that the needle tip is inserted to an intended depth. Disk 1380 includes a skin-contact surface 1383 that is in contact with the skin at the insertion site when acupuncture needle 1306 is fully inserted. In the illustrated embodiment, a ring-shaped electrode 1381 is incorporated onto skin-contact surface 1383. In one embodiment, an adhesive material 1382 is applied to skin-contact surface 1383, such as adjacent to electrode 1381, to allow disk 1380 to be stably attached onto the skin. In various embodiments, disk 1380 can include one or more electrodes of any suitable shape(s) incorporated onto skin-contact surface 1383. Handle 1355 includes an electrode connector 1356 that includes two connector portions that are insulated from each other. One of the two connector portions is electrically connected to needle body 1358, or electrically connected to needle body 1358 and handle 1355. The other of the two connector portions is electrically connected to electrode 1381. Electrode connector 1356 allow for separate electrical connections between needle body 1358 and one of IOD(s) 105 and between electrode 1381 and that IOD.

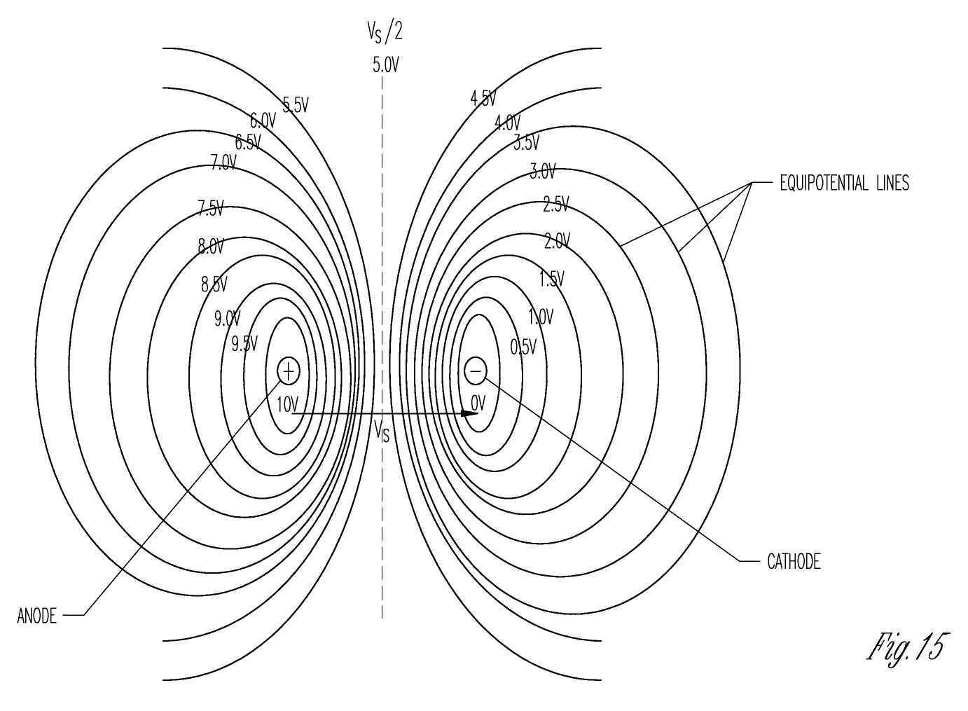

FIG. 14 is an illustration of an embodiment of acupuncture needle 1306 connected to medical device 1105 and inserted into the patient's tissue. Needle body 1358 and electrode 1381 are electrically connected to medical device 1005 via an electrode connector 1469-1 with a cable 1468-1 and an electrode connector 1469-2 with a cable 1468-2, respectively. As illustrated in FIG. 14, acupuncture needle 1306 provides a pair of electrodes needed to sense a signal from and/or deliver stimulation to target 1070, thereby eliminating the need for another acupuncture needle. Using a partially insulated acupuncture needle for acupuncture needle 1306 can provide the same advantages over using a non-insulated acupuncture needle as discussed above with reference to FIG. 10.

### Examples of Health State Detection

In various embodiments, the present multi-point stimulation and data collection system can be used to detect a health state (including physiological and/or pathological states) in a patient based on distribution of an externally applied electric field in the patient's body as measured from selected acupoints. In such embodiments, the stimulation may include non-excitatory stimulation that is not intended to excite tissue in the body. This is also referred to as sub-threshold stimulation because the stimulation intensity is to be set below the threshold above which action potentials will be evoked. This non-excitatory stimulation is applied to the patient to produce an electrical field whose distribution can be monitored and analyzed for detecting the health state of the patient.

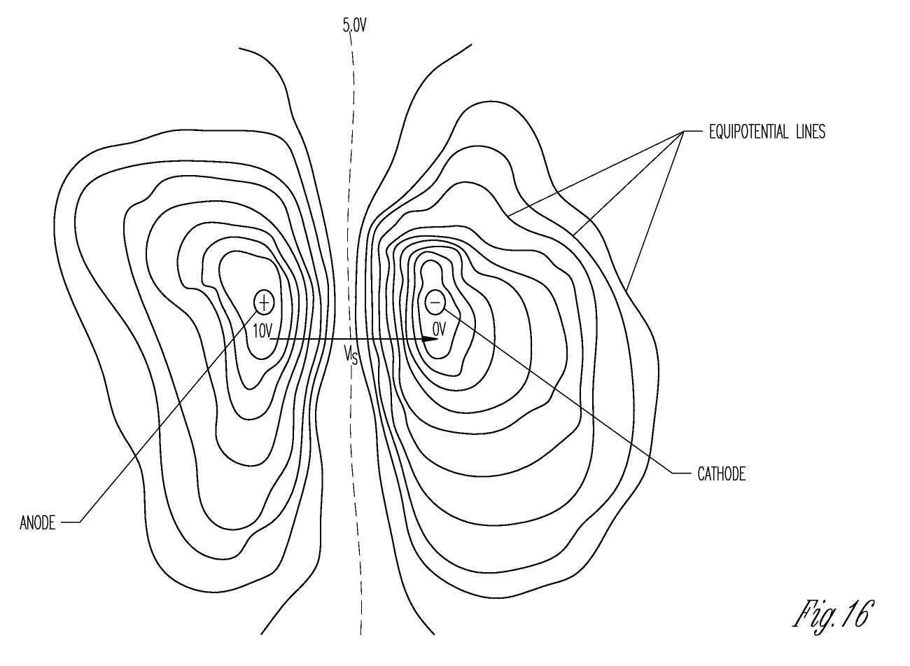

FIG. 15 is an illustration of an example of distribution of an electric field produced by a dipole in a uniform dielectric medium. The electric field is produced by applying a test signal via a pair of anode and cathode. The test signal can be a non-excitatory stimulation signal delivered from a voltage source and has an amplitude of V_{S} (the difference between the anodic and cathodic potentials). The current of the test signal flows from the anode to the cathode in directions (i.e., the direction of the electric field) perpendicular to the equipotential lines. The voltage amplitude of the test signal measured in the uniform dielectric medium approaches one half of V_{S} (V_{S}/2) as the point of measurement moves farther away from the pair of anode and cathode. Thus, the far field can be considered to be a uniform field with a constant intensity at V_{S}/2.

FIG. 16 is an illustration of an example of distribution of an electric field produced by a dipole in a non-uniform dielectric medium. A patient's body or various portions of interest in the patient's body can be considered to be a non-uniform dielectric medium, with a non-uniform electrical conductivity and a distribution of equipotential lines that is dependent on the structure of the tissue media and the location of the source producing the electric field. As a result, the electric field may be non-uniform within a certain area surrounding the anode and the cathode. However, the field still becomes uniform at sufficiently distant regions.

An example of non-uniform distribution of electric field generated intrinsically in the human body is the electrocardiographic (ECG) signal. During each cardiac cycle, cardiomyocytes are excited intrinsically in a specific order. The excitation of cardiomyocytes is similar to a transient formation of a dipole in the body. Many such dipoles result in a composite electric field that propagates on the surface of the body and can be sensed and recorded as ECG. An externally generated test signal may be delivered to the patient to form a dipole electric field that may propagate in the patient's body in a similar manner.

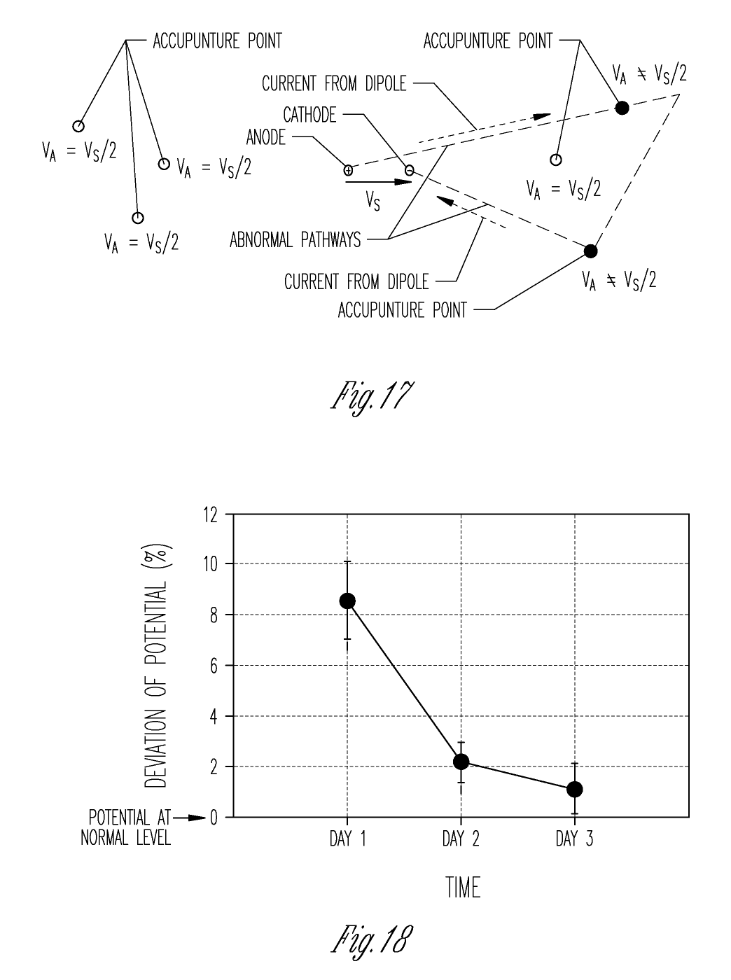

Characteristics of electric field resulting from an externally applied dipole and traveling through the meridian system can be used for various diagnostic purposes. FIG. 17 is an illustration of an example of electric field of a dipole in a diseased human body. Under sub-optimal or abnormal or diseased conditions, at least certain portions of the meridian system can fall into various states of dysfunction. To compensate for obstruction to the flow of qi, these portions of the meridian system undergo various changes, such as abnormally high level of cellular activities and edema. The responses at various acupoints in these portions of the meridian system to externally applied physical stimuli (e.g., electrical, acoustic, or thermal stimuli) become different from the responses at other unaffected acupoints. Under such circumstances, if a dipole is formed on or under the skin, some electrical current may take “short-circuited” pathways in the dysfunctional portions of the meridian system to reach distant acupoints, resulting in a substantially non-uniform propagation of the electric field of the dipole and deviation of the electric potentials measured from the distant acupoints. For example, as illustrated in FIG. 17, when a test signal having a voltage amplitude of V_{S} is applied via a dipole, the voltage V_{A} measured from acupoints that are sufficiently far from the dipole became substantially different from V_{S}/2.

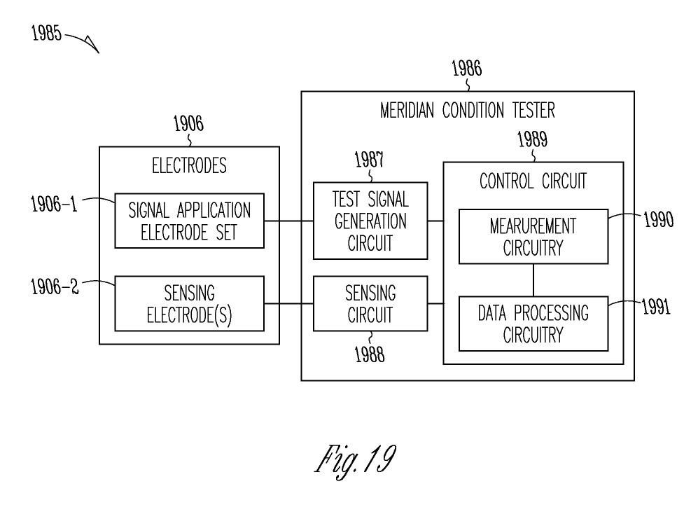

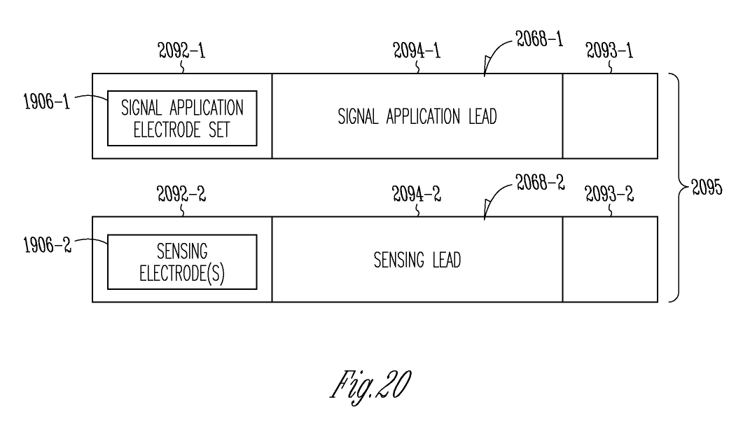

FIG. 18 is an illustration of an example of deviation of electric potentials measured from acupoints distant from a dipole in a patient. The patient suffered from dysmenorrhea and was treated with acupuncture for three days. An experiment of dipole field test as discussed above with reference to FIG. 18 was conducted during the treatment. The treatment included a 20-minute session of acupuncture therapy each day for the three days. Voltage measurements were performed at selected acupoints three times each day, before the acupuncture therapy, at 10 minutes into the acupuncture therapy, and at the end of the acupuncture therapy. The test signal has a voltage amplitude (V_{S}) of 0.008 V. The measured parameter included the deviation of the voltage V_{A} from its normal value (V_{S}/2). The graph shows this deviation expressed in percentage points, with the mean and variance from the three measurements each day. As shown in FIG. 18, the reduction in the deviation indicates the beneficial effect of the acupuncture therapy. In such ways, the present system can be used to verify a need to apply acupuncture therapy, to verify selection of acupoints for applying the acupuncture therapy, and to monitor an effect of the acupuncture therapy. This can be done, for example, using the stimulation and sensing capabilities of system 100, including its various embodiments such as those discussed below with reference to FIGS. 19-23.

FIG. 19 is a block diagram illustrating an embodiment of a system 1985 including a meridian condition tester 1986 and electrodes 1906 for detecting the health state of a patient. System 1985 can be implemented in system 100 or implemented as a stand-alone system.

Electrodes 1906 can include a signal application electrode set 1906-1 for applying a test signal to the patient and one or more sensing electrodes 1906-2 for sensing responses to the application of the test signal. In various embodiments, the test signal is applied as a form on non-excitatory (sub-threshold) stimulation. Signal application electrode set 1906-1 includes at least an anode and a cathode (e.g., as illustrated in FIGS. 15 and 16) to be placed in a signal application site on or in the patient to allow delivery of the test signal to the signal application site. Sensing electrode(s) 1906-2 are each to be placed in or on a sensing point selected from various acupoints of the patient. Examples for each electrode of electrodes 1906 include surface electrodes, such as skin patch electrodes, transcutaneous electrical nerve stimulation (TENS) electrodes, and surface neuromuscular sensing electrodes, and percutaneous electrodes, such as standard acupuncture needles and needle electrodes including, but not limited to the needle electrodes discussed above with reference to FIGS. 6-14 (e.g., electrodes 606, 706, 806, 906, 1006, 1306).

Meridian condition tester 1986 can include a test signal generation circuit 1987, a sensing circuit 1988, and a control circuit 1989. Test signal generation circuit 1987 can generate the test signal to be delivered to the patient through signal application electrode set 1906-1. The test signal has a test signal amplitude (V_{S}). Sensing circuit 1988 can sense one or more response signals each representative of propagation of the test signal to one of one or more sensing points. Each sensing point can be an acupoint. Control circuit 1989 can include measurement circuitry 1990 and data processing circuitry 1991. Measurement circuitry 1990 can measure a response to the delivery of the test signal. The response can include a voltage amplitude (V_{A}) of each sensed response signal. Data processing circuitry 1991 can determine a ratio of the V_{A} to the V_{S}, V_{A}/V_{S}, for each sensing point and can produce a meridian condition indicator based on the ratio for that sensing point.

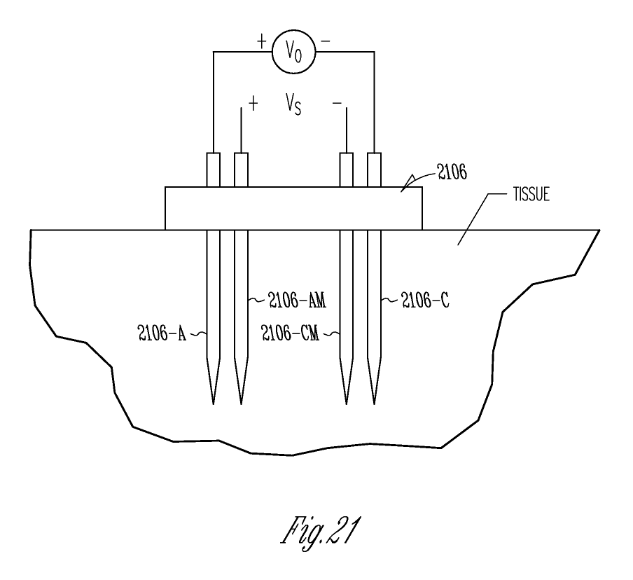

FIG. 20 is a block diagram illustrating an embodiment of electrodes with leads for detecting the health state of the patient, such as for use in system 1985. For purposes of illustration and discussion, FIG. 20 shows a lead system 2095 including a signal application lead 2068-1 and a sensing lead 2068-2. In various embodiment, lead system 2095 can include any number of signal application leads such as signal application lead 2068-1 and any number of sensing leads such as sensing lead 2068-2 as needed for the detection of the health state.

In the illustrated embodiment, signal application lead 2068-1 includes a distal end portion 2092-1, a proximal end portion 2093-1, and an elongate body 2094-1 coupled between distal end portion 2092-1 and proximal end portion 2093-1. Distal end portion 2092-1 includes, or is configured to be connected to, one or more electrodes of signal application electrode set 1906-1, including the anode and/or the cathode. In various embodiments, one or more signal application leads and signal application electrode set 1906-1 can be configured and/or connected as, for example, a lead with an anode and another lead with a cathode, a lead with both an anode and a cathode, a lead with multiple anodes and another lead with multiple cathodes, and a lead with multiple anodes and multiple cathodes. Proximal end portion 2093-1 can be configured for connection to meridian condition tester 1986. In various embodiments, signal application electrode set 1906-1 includes at least one pair of anode and cathode that form a dipole, and is configured such that when placed on or in the patient, the anode and the cathode are separated by a distance in a range of 10 and 500 mm, with approximately 80 mm being a specific example. In one example in which the states of various acupoints located in the leg region of the patient can be monitored and detected, the anode is to be placed near acupoint ST36 (Zusanli) and the cathode is to be placed near acupoint SP8 (Diji).

In various embodiments, criteria for choosing the signal application site (i.e., site for placing signal application electrode set 1906-1 for delivering the test signal) can include:
1) at least one of the anode and the cathode should be placed on a meridian path; and
2) given that the distance between the anode and the cathode is D (i.e., the length of the line between the anode and the cathode, such as between the geometric centers of the anode and the cathode),
   a. the distance between the midpoint between the anode and the cathode (i.e., the exact middle point of the line between the anode and the cathode) and the nearest sensing point equals or is longer than D, and
   b. the distance between the midpoint between the anode and the cathode and the farthest sensing point should equals or is shorter than 5D.
      According to 2), if the distance between the anode and the cathode is D, and the distances each between one of N sensing points and the midpoint between the anode and the cathode is dₙ, (n=1, 2, . . . N), than min (d₁, d₂, . . . d_{N})≧D and max (d₁, d₂, . . . d_{N})≦5D.

In the illustrated embodiment, sensing lead 2068-2 includes a distal end portion 2092-2, a proximal end portion 2093-2, and an elongate body 2094-2 coupled between distal end portion 2092-2 and proximal end portion 2093-2. Distal end portion 2092-2 includes, or is configured to be connected to, one or more sensing electrodes of sensing electrode(s) 1906-2. In various embodiments, one or more sensing leads and sensing electrode(s) 1906-2 can be configured and/or connected as, for example, a lead with a sensing electrode and, a lead with multiple sensing electrodes. Proximal end portion 2093-2 can be configured for connection to meridian condition tester 1986.

Each sensing electrode of sensing electrode(s) 1906-2 is to be placed in or on a sensing point. A “sensing point” as used in this document refers to a site in or on the patient, such as an acupoint, identified for placing a sensing electrode. One or more sensing points may be selected by the user (e.g., an acupuncture practitioner) based on known or believed relationship between a measurable state the acupoint and a specific medical condition. In some examples, an acupoint for treatment of a specific medical condition can be used as a sensing point for that specific medical condition.

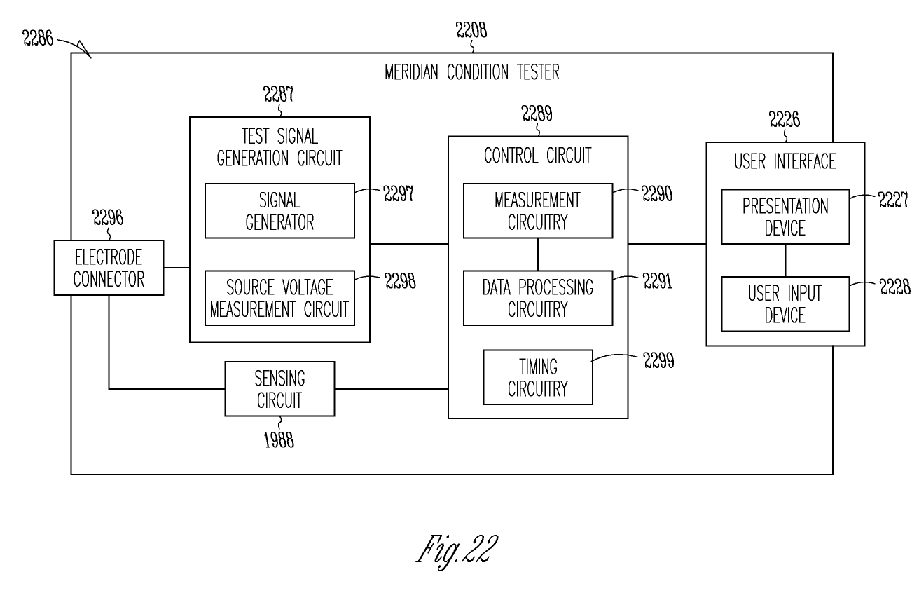

FIG. 21 is an illustration of an embodiment of electrodes 2106 for applying the test signal and measuring amplitude of the source voltage (i.e., the test signal amplitude, V_{S}), such as for use in system 1985. In some embodiments, source voltage measurement electrodes are placed adjacent to each of the anode and the cathode of signal application electrode set 1906-1 to allow for measurement of the amplitude of the test signal (V_{S}) as applied on tissue of the patient at the signal application site.

In the illustrated embodiment, electrodes 2106 are configured as an electrode array including an anode 2106-A, an anode voltage measurement electrode 2106-AM, a cathode 2106-C, and a cathode voltage measurement electrode 2106-CM. V_{S} can be measured at the tissue between the anode and the cathode using the source voltage measurement electrodes including anode voltage measurement electrode 2106-AM and cathode voltage measurement electrode 2106-CM. V_{O} is the voltage applied across the anode and cathode through a lead. The relationship between V_{S} and V_{O} is:

V_{S}=V_{O}−V_{LE}−V_{ET},

where V_{LE} is the total voltage drop over lead and electrode impedances, and V_{ET}: total voltage drop over electrode-tissue interface impedance. In practice, V_{S} as measured at the tissue can be substantially smaller than V_{O}. In various embodiments, the distance between anode 2106-A and anode voltage measurement electrode 2106-AM and the distance between cathode 2106-C and cathode voltage measurement electrode 2106-CM can each be 2 to 20 mm, with approximately 5 mm being a specific example.

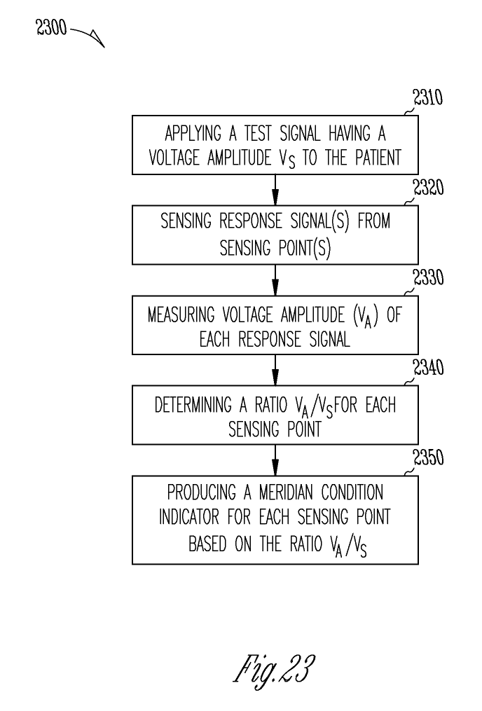

FIG. 22 is a block diagram illustrating an embodiment of a meridian condition tester 2286, which represents an example of meridian condition tester 1986. Meridian condition tester 2286 can collect meridian data indicative of a condition of the meridian system at one or more sensing points. Each sensing point may be selected from acupoints of the patient. The condition of the meridian system includes the electrical property indicative of a health state (such as a specific disease state) of the patient. In various applications, meridian condition tester 2286 when used with electrodes such as those discussed above in this document allows for monitoring of progress in the health state. Changes in the electrical property in response to a treatment indicates changes of the health state resulting from the treatment. Thus, meridian condition tester 2286 can be used for monitoring of effect of the treatment. In various embodiments, meridian condition tester 2286 can be an electronic device implemented in substation 303 and IOD 405 or implemented as a stand-alone device such as a portable device. An example of meridian condition tester 2286 has been implemented by Acumedical Incorporated, North Oaks, Minn., U.S.A., as a device known as AcuRanger™.

Meridian condition tester 2286 can include a user interface 2226, an electrode connector 2296, a test signal generation circuit 2287, a sensing circuit 1988, a control circuit 2289, and a chassis 2208. In the illustrated embodiment, user interface 2226 and electrode connector 2296 are incorporated into chassis 2208, which houses test signal generation circuit 2287, sensing circuit 1988, and control circuit 2289.

User interface 2226 can include a presentation device 2227 and a user input device 2228. In an embodiment in which meridian condition tester 2286 is implemented in substation 303 and IOD 405, user interface 2226 can be implemented as user interface 326 in substation 303, with presentation device 2227 implemented as presentation device 327 and user input device implemented as user input device 328. In an embodiment in which meridian condition tester 2286 is implemented as a stand-alone device, it can be figured, for example, to include all the features and perform all the functions as discussed above for user interface 326.

Electrode connector 2296 provides for electrical connections between the circuit of meridian condition tester 2286 and electrodes such as electrodes 1906. For example, proximal end portion 2093-1 of signal application lead 2068-1 and proximal end portion 2093-2 of sensing lead 2068-2 can each be configured to be connected with electrode connector 2296. In an embodiment in which meridian condition tester 2286 is implemented in substation 303 and IOD 405 electrode connector 2296 can be implemented as electrode connector 440 in IOD 405. In various embodiments, electrode connector 2296 can provide for an electrical connection between the circuit of meridian condition tester 2286 and each electrode directly or through a lead.

Test signal generation circuit 2287 represents an example of test signal generation circuit 1987. In an embodiment in which meridian condition tester 2286 is implemented in substation 303 and IOD 405, test signal generation circuit 2287 can be implemented as stimulation circuit 439 in IOD 405. In the illustrated embodiment, test signal generation circuit 2287 includes a signal generator 2297 and a source voltage measurement circuit 2298. Signal generator 2297 can generate the test signal. Examples of the test signal include an independent-voltage signal having a voltage output (V_{O}) not affected by tissue impedance and an independent-current signal having a current output that is not affected by tissue impedance. Examples of waveform types of the test signal include a sinusoidal wave, a square wave, a train of pulses, a saw tooth wave, a modification of any of the above waveforms, and a combination of any of the above waveforms. In various embodiments, the test signal can have any waveform compatible with signal application and sensing techniques discussed in this document. In some embodiments, signal generator 2297 can generate multiple test signals to be applied using multiple signal application electrode sets placed in different signal application sites in the same patient. In such embodiments, signal generator 2297 can deliver the multiple test signals to each set of the multiple signal application electrode sets independently and can control timing of delivery of each of the multiple test signals to allow for simultaneous, concurrent, or non-overlapping deliveries. Source voltage measurement circuit 2298 allows for measurement of the V_{S} using source voltage measurement electrodes such as anode voltage measurement electrode 2106-AM and cathode voltage measurement electrode 2106-CM when the test signal is applied using anode 2106-A and cathode 2106-C.

Sensing circuit 1988 can sense one or more response signals through one or more sensing electrodes such as sensing electrode(s) 1906-2. Each response signal is representative of propagation of the test signal to a sensing point. For each test signal delivered to a signal application site, multiple response signals can be sensed from multiple sensing points such as different acupoints. In an embodiment in which meridian condition tester 2286 is implemented in substation 303 and IOD 405, sensing circuit 1988 can be implemented as sensing circuit 438 in IOD 405.

Control circuit 2289 represents an example of control circuit 1989. In an embodiment in which meridian condition tester 2286 is implemented in substation 303 and IOD 405, control circuit 2289 can be implemented in control circuit 441 in IOD 405 and/or control circuit 329 in substation 303. In the illustrated embodiment, control circuit 2289 includes measurement circuitry 2290, timing circuitry 2299, and data processing circuitry 2291. Measurement circuitry 2290 can measure a response to the delivery of the test signal to the patient. The response can include a voltage amplitude (V_{A}) of each of the sensed one or more response signals. Timing circuitry 2299 can control timing of delivery of the test signal and the measurement of the response. Data processing circuitry 2291 can produce one or more meridian condition indicators using the measured response and present the one or more meridian condition indicators using presentation device 2227.

Examples of the one or more meridian condition indicators include a “field dispersion index” (FDI) and its variations and trends. The FDI can be defined as the absolute difference between the ratio of the measured voltage (V_{A}) to the test signal amplitude (V_{S}), V_{A}/V_{S}, and a reference value of 0.5, i.e.:

FDI=|V_{A}/V_{S}−0.5|.

FDI can be expressed in percentage points (ppt). When V_{A}/V_{S} and FDI are expressed as percentage points:

FDI(ppt)=|V_{A}/V_{S}(%)−50%|.

Data processing circuitry 2291 can determine the V_{A}/V_{S} and then calculate the FDI and present on presentation device 2227 one or more value of the FDI for each sensing point from which the response is measured using measurement circuitry 2290. The following are examples of FDIs and FDI trends that can be determined by data processing circuitry 2291 and presented using presentation device 2227 as needed:
a) FDI for a single acupoint during or after a treatment session;
b) an FDI trend for a single acupoint over multiple treatment sessions administered at different times;
c) FDIs for multiple acupoints during or after a treatment session;
d) FDI trends for multiple acupoints over multiple treatment sessions administered at different times;
e) a desired value range for the FDI in any of a)-d), e.g., displayed as a band;
f) an audial and/or visual indication when an FDI falls outside of a desired value range; and
g) an audial and/or visual guidance suggesting one or more acupoints that are most sensitive to changes in the health state for specific medical conditions.
   In various embodiments, the treatment sessions can include, but are not limited to, traditional Chinese medicine (TCM) treatment sessions. These TCM treatment sessions may include, but are not limited to, acupuncture therapies. The treatment sessions change electrical property of the meridian system. The changes can be quantitatively measured at the one or more sensing points.

FIG. 23 is a flow chart illustrating an embodiment of a method 2300 for detecting a health state of a patient. In one embodiment, method 2300 is performed by system 1985, includes various embodiments of its components as discussed in this document.

At 2310, a test signal is applied to the patient, the test signal having a test signal amplitude (V_{S}). In one embodiment, the test signal is delivered to the patient through a signal application electrode set (e.g., an electrode array) placed in a signal application site of the patient. The signal application electrode array includes at least an anode and a cathode forming a dipole. In one embodiment, The V_{S} is measured using source voltage measurement electrodes incorporated into the signal application electrode array and each placed adjacent to one of the anode and the cathode. In various embodiments, at least one of the anode and the cathode is placed on a meridian path of the meridian system of the patient.

At 2320, one or more response signals are sensed. The one or more response signals are each representative of propagation of the test signal to one of one or more sensing points selected from the acupoints of the patient. In various embodiments, the one or more response signals are sensed using one or more sensing electrodes each placed in a sensing point. Each sensing point is selected based on a relationship between a measurable state of that sensing point and a specific medical condition. In various embodiments, each sensing electrode is placed such that a distance between that sensing electrode and a midpoint between the anode and the cathode of the signal application electrode set is in a range from a minimum length to a maximum length. The minimum length equals to a distance between the anode and the cathode. The maximum length equals to five times of the distance between the anode and the cathode.

At 2330, a response to the delivery of the test signal is measured. The response includes a voltage amplitude (V_{A}) of each sensed response signal. At 2340, a ratio of the V_{A} to the V_{S}, V_{A}/V_{S}, is measured for each sensing point of the one or more sensing points;

At 2350, a meridian condition indicator is produced for the each sensing point based on the ratio. In various embodiments, one or more field dispersion indexes (FDIs) are each produced as the meridian condition indicator. The FDIs are each calculated as an absolute difference between the V_{A}/V_{S} ratio and a reference value of 0.5 (FDI=|V_{A}/V_{S}−0.5|) for a sensing point. In one embodiment, one or more FDI trends are each produced for a sensing point over multiple treatment sessions administered to the patient at different times. In one embodiment, FDIs are calculated for multiple sensing points during or after a treatment session administered to the patient. In various embodiments, one or more FDIs are presented with one or more desired ranges for each FDI. Delivery of therapy to the patient can be controlled based on the one or more FDIs and the one or more desired ranges. In various embodiments, values of each FDI can be presented in percentage points.

Some non-limiting examples of apparatuses and methods according to the present subject matter are provided as follows.

In Example 1, system for detecting a health state in a patient having a meridian system including acupoints may include a test signal generation circuit, a sensing circuit, and a control circuit. The test signal generation circuit may be configured to generate a test signal having a test signal amplitude (V_{S}) and to deliver the test signal to the patient. The sensing circuit may be configured to sense one or more response signals each representative of propagation of the test signal to one of one or more sensing points selected from the acupoints. The control circuit may be coupled to the test signal generation circuit and the sensing circuit, and may include measurement circuitry and data processing circuitry. The measurement circuitry may be configured to measure a response to the delivery of the test signal. The response may include a voltage amplitude (V_{A}) of each sensed response signal of the one or more sensed response signals. The data processing circuitry may be configured to determine a ratio of the V_{A} to the V_{S} (V_{A}/V_{S}) for each sensing point of the one or more sensing points and to produce a meridian condition indicator based on the V_{A}/V_{S}.

In Example 2, the subject matter of claim 1 may optionally be configured such that the data processing circuitry is configured to calculate one or more field dispersion indexes (FDIs) each being a function of the V_{A}/V_{S} for a sensing point of the one or more sensing points.

In Example 3, the subject matter of any one or any combination of Examples 1 and 2 may optionally be configured such that the data processing circuitry is configured to determine one or more FDI trends each being a trend of the FDI calculated for a sensing point of the one or more sensing points over multiple treatment sessions administered to the patient at different times.

In Example 4, the subject matter of Examples 2 or 3 may optionally be configured such that the data processing circuitry is configured to calculate the FDIs for multiple sensing points of the one or more sensing points during or after a treatment session administered to the patient.

In Example 5, the subject matter of any one or any combination of Examples 2 to 4 may optionally be configured to further include a user interface and optionally be configured such that the data processing circuitry is configured to present the one or more FDIs using the user interface.

In Example 6, the subject matter of claim 5 may optionally be configured such that the data processing circuitry is configured to present one or more desired ranges each for an FDI of the presented one or more FDIs using the user interface.

In Example 7, the subject matter of claim 6 may optionally be configured such that the data processing circuitry is configured to produce one or more of an audial indication or a visual indication when at least an FDI of the presented one or more FDIs falls outside of a corresponding desired range of the one or more desired ranges and to present the one or more of the audial indication or the visual indication using the user interface.

In Example 8, the subject matter of any one or any combination of Examples 1 to 7 may optionally be configured such that the data processing circuitry is configured to determine a guidance suggesting one or more acupoints that are most sensitive to changes in the health state of the patient for one or more specific physiological conditions.

In Example 9, the subject matter of any one or any combination of Examples 5 to 8 may optionally be configured to include a meridian condition tester. The meridian condition tester may include the test signal generation circuit, the sensing circuit, the control circuit, the user interface, and a chassis. The chassis houses the test signal generation circuit, the sensing circuit, and the control circuit. At least a portion of the user interface is incorporated into the chassis.

In Example 10, the subject matter of claim 9 may optionally be configured such that the meridian condition tester further includes an electrode connector incorporated into the chassis, and further comprising a signal application electrode set including at least an anode and a cathode and configured to be placed on the patient to allow delivery of the test signal from the test signal generation circuit to the patient.

In Example 11, the subject matter of claim 10 may optionally be configured such that the signal application electrode set further includes source voltage measurement electrodes each placed adjacent to one of the anode and the cathode, and configured to allow for measurement of a value of the V_{S} from the patient, and the data processing circuitry is configured to determine the V_{A}/V_{S} using the measured value of the V_{S}.

In Example 12, the subject matter of claim 11 may optionally be configured such that the test signal generation circuit includes a signal generator configured to generate the test signal, a source voltage measurement circuit configured to measure the value of the V_{S} using the source voltage measurement electrodes

In Example 13, the subject matter of any one or any combination of Examples 2 to 12 may optionally be configured such that the data processing circuitry is configured to calculate each FDI of the one or more FDIs as an absolute difference between the V_{A}/V_{S} and a reference value of 0.5 (FDI=|V_{A}/V_{S}×0.5|).

In Example 14, a method for detecting a health state in a patient having a meridian system including acupoints is provided. The method may include delivering a test signal to the patient, the test signal having a test signal amplitude (V_{S}), sensing one or more response signals each representative of propagation of the test signal to one of one or more sensing points selected from the acupoints, measuring a response to the delivery of the test signal, the response including a voltage amplitude (V_{A}) of each sensed response signal of the one or more sensed response signals, determining a ratio of the V_{A} to the V_{S} (V_{A}/V_{S}) for each sensing point of the one or more sensing points, and producing a meridian condition indicator for the each sensing point based on the ratio.

In Example 15, the subject matter of applying the test signal to the patient as found in Example 14 may optionally include delivering the test signal to the patient through a signal application electrode array placed in a signal application site of the patient. The signal application electrode array includes at least an anode and a cathode forming a dipole.

In Example 16, the subject matter of Example 15 may optionally further include measuring the V_{S} using source voltage measurement electrodes incorporated into the signal application electrode array and each placed adjacent to one of the anode and the cathode.

In Example 17, the subject matter of any one or any combination of Examples 15 and 16 may optionally further include placing at least one of the anode and the cathode on a meridian path of the meridian system.

In Example 18, the subject matter of sensing the one or more response signals as found in any one or any combination of Examples 14 to 17 may optionally include sensing the one or more response signals using one or more sensing electrodes each placed in one of the one or more sensing points, and the subject matter of any one or any combination of Examples 14 to 17 may further include selecting each sensing point of the one or more sensing points based on a relationship between a measurable state of the each sensing point and a specific condition related to the patient's health.

In Example 19, the subject matter of Example 18 may optionally further include placing each sensing electrode of the one or more sensing electrodes such that a distance between the each sensing electrode and a midpoint between the anode and the cathode is in a range from a minimum length equaling to a distance between the anode and the cathode to a maximum length equaling to five times of the distance between the anode and the cathode.

In Example 20, the subject matter of producing the meridian condition indicator as found in any one or any combination of Examples 14 to 19 may optionally include calculating one or more field dispersion indexes (FDIs) each being a function of the V_{A}/V_{S} for a sensing point of the one or more sensing points.

In Example 21, the subject matter of producing the meridian condition indicator as found in Example 20 may optionally include determining one or more FDI trends each being a trend of the FDI calculated for a sensing point of the one or more sensing points over multiple treatment sessions administered to the patient at different times.

In Example 22, the subject matter of producing the meridian condition indicator as found in any one or any combination of Examples 20 and 21 may optionally include calculating the FDIs for multiple sensing points of the one or more sensing points during or after a treatment session administered to the patient.

In Example 23, the subject matter of any one or any combination of Examples 20 to 22 may optionally further include presenting the one or more FDIs with one or more desired ranges each for an FDI of the presented one or more FDIs.

In Example 24, the subject matter of any one or any combination of Examples 20 to 23 may optionally further include controlling a delivery of therapy to the patient based on the one or more FDIs and the one or more desired ranges.

In Example 25, the subject matter of calculating the one or more FDIs as found in any one or any combination of Examples 20 to 24 may optionally include calculating each FDI of the one or more FDIs as an absolute difference between the V_{A}/V_{S} and a reference value of 0.5 (FDI=|V_{A}/V_{S}−0.5|).

In Example 26, the subject matter of Example 25 may optionally include presenting the one or more FDI in percentage points.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for detecting a health state in a patient having a meridian system including acupoints, the system comprising:
a test signal generation circuit configured to generate a test signal having a test signal amplitude (V_{S}) and to deliver the test signal to the patient;
a sensing circuit configured to sense one or more response signals each representative of propagation of the test signal to one of one or more sensing points selected from the acupoints; and
a control circuit coupled to the test signal generation circuit and the sensing circuit, the control circuit including:
measurement circuitry configured to measure a response to the delivery of the test signal, the response including a voltage amplitude (V_{A}) of each sensed response signal of the one or more sensed response signals; and
data processing circuitry configured to determine a ratio of the V_{A} to the V_{S} (V_{A}/V_{S}) for each sensing point of the one or more sensing points and to produce a meridian condition indicator based on the V_{A}/V_{S}.

2. The system of claim 1, wherein the data processing circuitry is configured to calculate one or more field dispersion indexes (FDIs) each being a function of the V_{A}/V_{S} for a sensing point of the one or more sensing points.

3. The system of claim 2, wherein the data processing circuitry is configured to determine one or more FDI trends each being a trend of the FDI calculated for a sensing point of the one or more sensing points over multiple treatment sessions administered to the patient at different times.

4. The system of claim 2, wherein the data processing circuitry is configured to calculate the FDIs for multiple sensing points of the one or more sensing points during or after a treatment session administered to the patient.

5. The system of claim 2, further comprising a user interface, and wherein the data processing circuitry is configured to present the one or more FDIs using the user interface.

6. The system of claim 5, wherein the data processing circuitry is configured to present one or more desired ranges each for an FDI of the presented one or more FDIs using the user interface.

7. The system of claim 6, wherein the data processing circuitry is configured to produce one or more of an audial indication or a visual indication when at least an FDI of the presented one or more FDIs falls outside of a corresponding desired range of the one or more desired ranges and to present the one or more of the audial indication or the visual indication using the user interface.

8. The system of claim 6, wherein the data processing circuitry is configured to determine a guidance suggesting one or more acupoints that are most sensitive to changes in the health state of the patient for one or more specific medical conditions.

9. The system of claim 5, comprising a meridian condition tester including the test signal generation circuit, the sensing circuit, the control circuit, the user interface, and a chassis housing the test signal generation circuit, the sensing circuit, and the control circuit, wherein at least a portion of the user interface is incorporated into the chassis.

10. The system of claim 9, wherein the meridian condition tester further comprises an electrode connector incorporated into the chassis, and further comprising a signal application electrode set including at least an anode and a cathode and configured to be placed on the patient to allow delivery of the test signal from the test signal generation circuit to the patient.

11. The system of claim 10, wherein the signal application electrode set further comprises source voltage measurement electrodes each placed adjacent to one of the anode and the cathode and configured to allow for measurement of a value of the V_{S} from the patient, and the data processing circuitry is configured to determine the V_{A}/V_{S} using the measured value of the V_{S}.

12. The system of claim 11, wherein the test signal generation circuit comprises:
a signal generator configured to generate the test signal; and
a source voltage measurement circuit configured to measure the value of the V_{S} using the source voltage measurement electrodes.

13. The system of claim 2, wherein the data processing circuitry is configured to calculate each FDI of the one or more FDIs as an absolute difference between the V_{A}/V_{S} and a reference value of 0.5 (FDI=|V_{A}/V_{S}−0.5|).

14. A method for detecting a health state in a patient having a meridian system including acupoints, the method comprising:
applying a test signal to the patient, the test signal having a test signal amplitude (V_{S});
sensing one or more response signals each representative of propagation of the test signal to one of one or more sensing points selected from the acupoints;
measuring a response to the delivery of the test signal, the response including a voltage amplitude (V_{A}) of each sensed response signal of the one or more sensed response signals;
determining a ratio of the V_{A} to the V_{S} (V_{A}/V_{S}) for each sensing point of the one or more sensing points; and
producing a meridian condition indicator for the each sensing point based on the V_{A}/V_{S}.

15. The method of claim 14, wherein applying the test signal to the patient comprises delivering the test signal to the patient through a signal application electrode array placed in a signal application site of the patient, the signal application electrode array including at least an anode and a cathode forming a dipole.

16. The method of claim 15, further comprising measuring the V_{S} using source voltage measurement electrodes incorporated into the signal application electrode array and each placed adjacent to one of the anode and the cathode.

17. The method of claim 15, further comprising placing at least one of the anode and the cathode on a meridian path of the meridian system.

18. The method of claim 17, wherein sensing the one or more response signals comprises sensing the one or more response signals using one or more sensing electrodes each placed in one of the one or more sensing points, and further comprising selecting each sensing point of the one or more sensing points based on a relationship between a measurable state of the each sensing point and a specific medical condition related to the patient's health.

19. The method of claim 18, further comprising placing each sensing electrode of the one or more sensing electrodes such that a distance between the each sensing electrode and a midpoint between the anode and the cathode is in a range from a minimum length equaling to a distance between the anode and the cathode to a maximum length equaling to five times of the distance between the anode and the cathode.

20. The method of claim 14, wherein producing the meridian condition indicator comprises calculating one or more field dispersion indexes (FDIs) each being a function of the V_{A}/V_{S} for a sensing point of the one or more sensing points.

21. The method of claim 20, wherein producing the meridian condition indicator further comprises determining one or more FDI trends each being a trend of the FDI calculated for a sensing point of the one or more sensing points over multiple treatment sessions administered to the patient at different times.

22. The method of claim 20, wherein producing the meridian condition indicator further comprises calculating the FDIs for multiple sensing points of the one or more sensing points during or after a treatment session administered to the patient.

23. The method of claim 22, further comprising presenting the one or more FDIs with one or more desired ranges each for an FDI of the presented one or more FDIs.

24. The method of claim 23, further comprising controlling a delivery of therapy to the patient based on the one or more FDIs and the one or more desired ranges.

25. The method of claim 20, wherein calculating the one or more FDIs comprises calculating each FDI of the one or more FDIs as an absolute difference between the V_{A}/V_{S} and a reference value of 0.5 (FDI=|V_{A}/V_{S}−0.5|).

26. The method of claim 25, further comprising presenting the one or more FDI in percentage points.
